SEQUENCE LISTING (22 sequences)

SEQ ID NO: 1 - DNA, 2036 bp, Homo Sapiens
atgtactcggggaaccgcagcggcggccacggctactgggacggcggcggggccgcgggcgctaaggggccggcgccggcggggacactgagccccgcgcccctcttcagccccggcacctacgagcgcctggcgctgctgctgggctccattgggctgctgggcgtcggcaacaacctgctggtgctcgtcctctactacaagttccagcggctccgcactcccactcacctcctcctggtcaacatcagcctcagcgacctgctggtgtccctcttcggggtcacctttaccttcgtgtcctgcctgaggaacggctgggtgtgggacaccgtgggctgcgtgtgggacgggtttagcggcagcctcttcgggattgtttccattgccaccctaaccgtgctggcctatgaacgttacattcgcgtggtccatgccagagtgatcaatttttcctgggcctggagggccattacctacatctggctctactcactggcgtgggcaggagcacctctcctgggatggaacaggtacatcctggacgtacacggactaggctgcactgtggactggaaatccaaggatgccaacgattcctcctttgtgcttttcttatttcttggctgcctggtggtgcccctgggtgtcatagcccattgctatggccatattctatattccattcgaatgcttcgttgtgtggaagatcttcagacaattcaagtgatcaagattttaaaatatgaaaagaaactggccaaaatgtgctttttaatgatattcaccttcctggtctgttggatgccttatatcgtgatctgcttcttggtggttaatggtcatggtcacctggtcactccaacaatatctattgtttcgtacctctttgctaaatcgaacactgtatacaatccagtgatttatgtcttcatgatcagaaagtttcgaagatcccttttgcagcttctgtgcctccgactgctgaggtgccagaggcctgctaaagacctaccagcagctggaagtgaaatgcagatcagacccattgtgatgtcacagaaagatggggacaggccaaagaaaaaagtgactttcaactcttcttccatcatttttatcggcaccagtgatgaatcactgtcagttgacgacagcgacaaaaccaatgggtccaaagttgatgtaatccaagttcgtcctttgtaggaatgaagaatggcaacgaaagatggggccttaaattggatgccacttttggactttcatcataagaagtgtctggaatacccgttctatgtaatatcaacagaaccttgtggtccagcaggaaatccgaattgcccatatgctcttgggcctcaggaagaggttgaacaaaaacaaattcttttaattcaacgggtgctttacataatgaaaaaaccacttgtggcacacgatgggcatctaacatcatcatcttctaatgtgttggagattttcatttcaaatatattttttaaattactctattttccaaaacacgtaatgcatttttctcgaaaataccttactgtaaaaataactgtcgcgtacacatgtgtgaagtagctagaacatactgaattttttttgtactgttggactctattcagtgtcatgtcctatatctgatcaagttatcaaggagataattctagaatgaaaaagaaaatcctcttggtggaaccaaaagacgttttatatgtgcagtatgacaaagaggagtttcagagacaactttgaatccttgtcagcctggagaccagcaccagaggaatctacaaggcaaactcccatatatttgcttcccccaaattgctgcccctacagactcaaagctctttttctttgttttgttgtttctctaaaaatttactgttctttgtcgatgctatataagccagggagttctaagacgccagctctttgagatttgctcattcccctgtatttcccacatatatattacatatacccgctaataaatttatgtttgtttttaaaaatgtgtc

SEQ ID NO: 2 - protein, 402 aa, Homo Sapiens
MYSGNRSGGHGYWDGGGAAGAKGPAPAGTLSPAPLFSPGTYERLALLLGSIGLLGVGNNLLVLVLYYKFQRLRTPTHLLLVNISLSDLLVSLFGVTFTFVSCLRNGWVWDTVGCVWDGFSGSLFGIVSIATLTVLAYERYIRVVHARVINFSWAWRAITYIWLYSLAWAGAPLLGWNRYILDVHGLGCTVDWKSKDANDSSFVLFLFLGCLVVPLGVIAHCYGHILYSIRMLRCVEDLQTIQVIKILKYEKKLAKMCFLMIFTFLVCWMPYIVICFLVVNGHGHLVTPTISIVSYLFAKSNTVYNPVIYVFMIRKFRRSLLQLLCLRLLRCQRPAKDLPAAGSEMQIRPIVMSQKDGDRPKKKVTFNSSSIIFIGTSDESLSVDDSDKTNGSKVDVIQVRPL

SEQ ID NO: 3 - DNA, 619 bp, Homo Sapiens
  misc_feature (1)...(619): n = A,T,C or G
agcgcgccgcgagccccgccgcaagctgagcgcctccgcccgccaggcgcgccggcgccgggccatgtactcggggaaccgcagcggcggccacggctactgggacggcggcggggccgcgggcgctgaggggccggcgccggcggggacactgagccccgcgcccctcttcagccccggcacctacgagcgcctggcgctgctgctgggctccattgggctgctgggcgtcggcaacaacctgctggtgctcgtcctctactacaagttccagcggctccgcactcccactcacctcctcctggtcaacatcagcctcagcgacctgctggtgtccctcttcggggtcacctttaccttcgtgtcctgcctgaggaacggctgggtgtgggacaccgtgggctgcgtgtgggacgggtttagcggcagcctcttcgnttcgttgtgtggaagatcttcagacaattcaagtgatcaagattttaaaatatgaaaagaaactggccaaatgtgctttttaatgatattaccttcctggtctgttggatgccttatatcgtgatctgcttcttggtgggttaatggtattggtcacctggtactccaacaatatctattg

SEQ ID NO: 4 - protein, 163 aa, Homo Sapiens
  VARIANT (1)...(163): Xaa = Any Amino Acid
ARREPRRKLSASARQARRRRAMYSGNRSGGHGYWDGGGAAGAEGPAPAGTLSPAPLFSPGTYERLALLLGSIGLLGVGNNLLVLVLYYKFQRLRTPTHLLLVNISLSDLLVSLFGVTFTFVSCLRNGWVWDTVGCVWDGFSGSLFXSLCGRSSDNSSDQDFKI

SEQ ID NO: 5 - DNA, 1036 bp, Homo Sapiens
gaagggctgatgactttcagaagatgaaggtaagtagaaaccgttgatgggactgagaaaccagagttaaaacctctttggagcttctgaggactcagctggaaccaacgggcacagttggcaacaccatcatgacatcacaacctgttcccaatgagaccatcatagtgctcccatcaaatgtcatcaacttctcccaagcagagaaacccgaacccaccaaccaggggcaggatagcctgaagaaacatctacacgcagaaatcaaagttattgggactatccagatcttgtgtggcatgatggtattgagcttggggatcattttggcatctgcttccttctctccaaattttacccaagtgacttctacactgttgaactctgcttacccattcataggaccctttttttttatcatctctggctctctatcaatcgccacagagaaaaggttaaccaagcttttggtgcatagcagcctggttggaagcattctgagtgctctgtctgccctggtgggtttcattatcctgtctgtcaaacaggccaccttaaatcctgcctcactgcagtgtgagttggacaaaaataatataccaacaagaagttatgtttcttacttttatcatgattcactttataccacggactgctatacagccaaagccagtctggctggaactctctctctgatgctgatttgcactctgctggaattctgcctagctgtgctcactgctgtgctgcggtggaaacaggcttactctgacttccctgggagtgtacttttcctgcctcacagttacattggtaattctggcatgtcctcaaaaatgactcatgactgtggatatgaagaactattgacttcttaagaaaaaagggagaaatattaatcagaaagttgattcttatgataatatggaaaagttaaccattatagaaaagcaaagcttgagtttcctaaatgtaagcttttaaagtaatgaacattaaaaaaaaccattatttcactgtcaaaaaaaaaaaaaaa

SEQ ID NO: 6 - protein, 248 aa, Homo Sapiens
MTSQPVPNETIIVLPSNVINFSQAEKPEPTNQGQDSLKKHLHAEIKVIGTIQILCGMMVLSLGIILASASFSPNFTQVTSTLLNSAYPFIGPFFFIISGSLSIATEKRLTKLLVHSSLVGSILSALSALVGFIILSVKQATLNPASLQCELDKNNIPTRSYVSYFYHDSLYTTDCYTAKASLAGTLSLMLICTLLEFCLAVLTAVLRWKQAYSDFPGSVLFLPHSYIGNSGMSSKMTHDCGYEELLTS

SEQ ID NO: 7 - DNA, 932 bp, Homo Sapiens
gaagggctgatgactttcagaagatgaaggtaagtagaaaccgttgatgggactgagaaaccagagttaaaacctctttggagcttctgaggactcagctggaaccaacgggcacagttggcaacaccatcatgacatcacaacctgttcccaatgagaccatcatagtgctcccatcaaatgtcatcaacttctcccaagcagagaaacccgaacccaccaaccaggggcaggatagcctgaagaaacatctacacgcagaaatcaaagttattgggactatccagatcttgtgtggcatgatggtattgagcttggggatcattttggcatctgcttccttctctccaaattttacccaagtgacttctacactgttgaactctgcttacccattcataggaccctttttttttatcatctctggctctctatcaatcgccacagagaaaaggttaaccaagcttttggtgcatagcagcctggttggaagcattctgagtgctctgtctgccctggtgggtttcattatcctgtctgtcaaacaggccaccttaaatcctgcctcactgcagtggaactctctctctgatgctgatttgcactctgctggaattctgcctagctgtgctcactgctgtgctgcggtggaaacaggcttactctgacttccctgggagtgtacttttcctgcctcacagttacattggtaattctggcatgtcctcaaaaatgactcatgactgtggatatgaagaactattgacttcttaagaaaaaagggagaaatattaatcagaaagttgattcttatgataatatggaaaagttaaccattatagaaaagcaaagcttgagtttcctaaatgtaagcttttaaagtaatgaacattaaaaaaaaccattatttcactgtcaaaaaaaaaaaaaaa

SEQ ID NO: 8 - protein, 178 aa, Homo Sapiens
MTSQPVPNETIIVLPSNVINFSQAEKPEPTNQGQDSLKKHLHAEIKVIGTIQILCGMMVLSLGIILASASFSPNFTQVTSTLLNSAYPFIGPFFFIISGSLSIATEKRLTKLLVHSSLVGSILSALSALVGFIILSVKQATLNPASLQWNSLSDADLHSAGILPSCAHCCAAVETGLL

SEQ ID NO: 9 - DNA, 1758 bp, Homo Sapiens
atgccgccccgccgcccgtgggacagagaggctggcacgttgcaggtcctgggagcgctggctgtgctgtggctgggctccgtggctcttatctgcctcctgtggcaagtgccccgtcctcccacctggggccaggtgcagcccaaggacgtgcccaggtcctgggagcatggctccagcccagcttgggagcccctggaagcagaggccaggcagcagagggactcctgccagcttgtccttgtggaaagcatcccccaggacctgccatctgcagccggcagcccctctgcccagcctctgggccaggcctggctgcagctgctggacactgcccaggagagcgtccacgtggcttcatactactggtccctcacagggcctgacatcggggtcaacgactcgtcttcccagctgggagaggctcttctgcagaagctgcagcagctgctgggcaggaacatttccctggctgtggccaccagcagcccgacactggccaggacatccaccgacctgcaggttctggctgcccgaggtgcccatgtacgacaggtgcccatggggcggctcaccaggggtgttttgcactccaaattctgggttgtggatggacggcacatatacatgggcagtgccaacatggactggcggtctctgacgcaggtgaaggagcttggcgctgtcatctataactgcagccacctggcccaagacctggagaagaccttccagacctactgggtactgggggtgcccaaggctgtcctccccaaaacctggcctcagaacttctcatctcacttcaaccgtttccagcccttccacggcctctttgatggggtgcccaccactgcctacttctcagcgtcgccaccagcactctgtccccagggccgcacccgggacctggaggcgctgctggcggtgatggggagcgcccaggagttcatctatgcctccgtgatggagtatttccccaccacgcgcttcagccaccccccgaggtactggccggtgctggacaacgcgctgcgggcggcagccttcggcaagggcgtgcgcgtgcgcctgctggtcggctgcggactcaacacggaccccaccatgttcccctacctgcggtccctgcaggcgctcagcaaccccgcggccaacgtctctgtggacgtgaaagtcttcatcgtgccggtggggaaccattccaacatcccattcagcagggtgaaccacagcaagttcatggtcacggagaaggcagcctacataggcacctccaactggtcggaggattacttcagcagcacggcgggggtgggcttggtggtcacccagagccctggcgcgcagcccgcgggggccacggtgcaggagcagctgcggcagctctttgagcgggactggagttcgcgctacgccgtcggcctggacggacaggctccgggccaggactgcgtttggcagggctgaggggggcctctttttctctcggcgaccccgccccgcacgcgccctcccctctgaccccggcctgggcttcagccgcttcctcccgcaagcagcccgggtccgcactgcgccaggagccgcctgcgaccgcccgggcgtcgcaaaccgcccgcctgctctctgatttccgagtccagccccccctgagccccacctcctccagggagccctccaggaagccccttccctgactcctggcccacaggccaggcctaaaaaaaactcgtggcttcaaaaaaaaaaaaaaaa

SEQ ID NO: 10 - protein, 489 aa, Homo Sapiens
MPPRRPWDREAGTLQVLGALAVLWLGSVALICLLWQVPRPPTWGQVQPKDVPRSWEHGSSPAWEPLEAEARQQRDSCQLVLVESIPQDLPSAAGSPSAQPLGQAWLQLLDTAQESVHVASYYWSLTGPDIGVNDSSSQLGEALLQKLQQLLGRNISLAVATSSPTLARTSTDLQVLAARGAHVRQVPMGRLTRGVLHSKFWVVDGRHIYMGSANMDWRSLTQVKELGAVIYNCSHLAQDLEKTFQTYWVLGVPKAVLPKTWPQNFSSHFNRFQPFHGLFDGVPTTAYFSASPPALCPQGRTRDLEALLAVMGSAQEFIYASVMEYFPTTRFSHPPRYWPVLDNALRAAAFGKGVRVRLLVGCGLNTDPTMFPYLRSLQALSNPAANVSVDVKVFIVPVGNHSNIPFSRVNHSKFMVTEKAAYIGTSNWSEDYFSSTAGVGLVVTQSPGAQPAGATVQEQLRQLFERDWSSRYAVGLDGQAPGQDCVWQG

SEQ ID NO: 11 - DNA, 2852 bp, Homo Sapiens
atggtcatgaggcccctgtggagtctgcttctctgggaagccctacttcccattacagttactggtgcccaagtgctgagcaaagtcgggggctcggtgctgctggtggcagcgcgtccccctggcttccaagtccgtgaggctatctggcgatctctctggccttcagaagagctcctggccacgtttttccgaggctccctggagactctgtaccattcccgcttcctgggccgagcccagctacacagcaacctcagcctggagctcgggccgctggagtctggagacagcggcaacttctccgtgttgatggtggacacaaggggccagccctggacccagaccctccagctcaaggtgtacgatgcagtgcccaggcccgtggtacaagtgttcattgctgtagaaagggatgctcagccctccaagacctgccaggttttcttgtcctgttgggcccccaacatcagcgaaataacctatagctggcgacgggagacaaccatggactttggtatggaaccacacaggctcttcacagacggacaggtgctgagcatttccctgggaccaggagacagagatgtggcctattcctgcattgtctccaaccctgtcagctgggacttggccacagtcacgccctgggatagctgtcatcatgaggcagcaccagggaaggcctcctacaaagatgtgctgctggtggtggtgcctgtctcgctgctcctgatgctggttactctcttctctgcctggcactggtgcccctgctcagggaaaaagaaaaaggatgtccatgctgacagagtgggtccagagacagagaacccccttgtgcaggatctgccataaaggacaatatgaactgatgcctggactatcagtaaccccactgcacaggcacacgatgctctgggacataactggtgcctggaaatcaccatggtcctcatatctcccatgggaatcctgtcctgcctcgaaggagcagcctgggcagccatcacaccacgaggacaggaagcaccagcacgtttcacacctcccccttccctctcccatcttctcatatcctggctcttctctgggcaagatgagccaagcagaacattccatccaggacactggaagttctccaggatccagatccatggggacattaatagtccaaggcattccctcccccaccactattcataaagtattaaccaactggcaccaaggaattgcctccagcctgagtcctaggctctaaaagatattacatatttgaactaatagaggaactctgagtcacccatgccagcatcagcttcagccccagaccctgcagtttgagatctgatgcttcctgagggccaaggcattgctgtaagaaaaggtctagaaataggtgaaagtgagaggtgggggacaggggtttctctttctggcctaaggactttcaggtaatcagagttcatgggccctcaaaggtaaattgcagttgtagacaccgaggatggttgacaacccatggttgagatgggcaccgttttgcaggaaacaccatattaatagacatcctcaccatctccatccgctctcacgcctcctgcaggatctgggagtgagggtggagagtctttcctcacgctccagcacagtggccaggaaaagaaatactgaatttgccccagccaacaggacgttcttgcacaacttcaagaaaagcagctcagctcaggatgagtcttcctgcctgaaactgagagagtgaagaaccataaaacgctatgcagaaggaacattatggagagaaagggtactgaggcactctagaatctgccacattcattttcaaatgcaaatgcagaagacttaccttagttcaaggggaggggacaaagaccccacagcccaacagcaggactgtagaggtcactctgactccatcaaactttttattgtggccatcttaggaaaatacattctgcccctgaatgattctgtctagaaaagctctggagtattgatcactactggaaaaacacttaaggagctaaacttaccttcggggattattagctgataaggttcacagtttctctcacccaggtgtaactggattttttctggggcctcaatccagtcttgataacagcgaggaaagaggtattgaagaaacaggggtgggtttgaagtactattttcccagggtggcttcaatctccccacctaggatgtcagccctgtccaaggaccttccctcttctccccagttcctgggcaatcacttcaccttggacaaaggatcagcacagctggcctccagatccacatcaccactcttccactcgattgttcccagatcctccctgcctggcctgctcagaggttccctgttggtaacctggctttatcaaattctcatccctttcccacacccacttctctcctatcaccttcccccaagattacctgaacagggtccatggccactcaacctgtcagcttgcaccatccccacctgccacctacagtcaggccacatgcctggtcactgaatcatgcaaaactggcctcagtccctaaaaatgatgtggaaaggaaagcccaggatctgacaatgagccctggtggatttgtggggaaaaaatacacagcactccccacctttctttcgttcatctccagggccccacctcagatcaaagcagctctggatgagatgggacctgcagctctccctccacaaggtgactcttagcaacctcatttcgacagtggtttgtagcgtggtgcaccagggccttgttgaacagatccacactgctctaataaagttcccatccttaatgaaaacc

SEQ ID NO: 12 - protein, 285 aa, Homo Sapiens
MVMRPLWSLLLWEALLPITVTGAQVLSKVGGSVLLVAARPPGFQVREAIWRSLWPSEELLATFFRGSLETLYHSRFLGRAQLHSNLSLELGPLESGDSGNFSVLMVDTRGQPWTQTLQLKVYDAVPRPVVQVFIAVERDAQPSKTCQVFLSCWAPNISEITYSWRRETTMDFGMEPHRLFTDGQVLSISLGPGDRDVAYSCIVSNPVSWDLATVTPWDSCHHEAAPGKASYKDVLLVVVPVSLLLMLVTLFSAWHWCPCSGKKKKDVHADRVGPETENPLVQDLP

SEQ ID NO: 13 - DNA, 1685 bp, Homo Sapiens
tcgctcgcagtccccttgccgacccgccggttttcttgtcctgttgggcccccaacatcagcgaaataacctatagctggcgacgggagacaaccatggactttggtatggaaccacacaggctcttcacagacggacaggtgctgagcatttccctgggaccaggagacagagatgtggcctattcctgcattgtctccaaccctgtcagctgggacttggccacagtcacgccctgggatagctgtcatcatgaggcagcaccagggaaggcctcctacaaagatgtgctgctggtggtggtgcctgtctcgctgctcctgatgctggttactctcttctctgcctggcactggtgcccctgctcaggaaacaccatattaatagacatcctcaccatctccatccgctctcacgcctcctgcaggatctgggagtgagggtggagagtctttcctcacgctccagcacagtggccaggaaaagaaatactgaatttgccccagccaacaggacgttcttgcacaacttcaagaaaagcagctcagctcaggatgagtcttcctgcctgaaactgagagagtgaagaaccataaaacgctatgcagaaggaacattatggagagaaagggtactgaggcactctagaatctgccacattcattttcaaatgcaaatgcagaagacttaccttagttcaaggggaggggacaaagaccccacagcccaacagcaggactgtagaggtcactctgactccatcaaactttttattgtggccatcttaggaaaatacattctgcccctgaatgattctgtctagaaaagctctggagtattgatcactactggaaaaacacttaaggagctaaacttagcttcggggattattagctgataaggttcacagtttctctcacccaggtgtaactggattttttctggggcctcaatccagtcttgataacagcgaggaaagaggtattgaagaaacaggggtgggtttgaagtactattttcccagggtggcttcaatctccccacctaggatgtcagccctgtccaaggaccttccctcttctccccagttcctgggcaatcacttcaccttggacaaaggatcagcacagctggcctccagatccacatcaccactcttccactcgattgttcccagatcctccctgcctggcctgctcagaggttccctgttggtaacctggctttatcaaattctcatccctttcccacacccacttctctcctatcaccttcccccaagattacctgaacagggtccatggccactcaacctgtcagcttgcaccatccccacctgccacctacagtcaggccacatgcctggtcactgaatcatgcaaaactggcctcagtccctaaaaatgatgtggaaaggaaagcccaggatctgacaatgagccctggtggatttgtggggaaaaaatacacagcactccccacctttctttcgttcatctccagggccccacctcagatcaaagcagctctggatgagatgggacctgcagctctccctccacaaggtgactcttagcaacctcatttcgacagtggtttgtagcgtggtgcaccagggccttgttgaacagatccacactgctctaataaagttcccatccttaaaaaaaaaaaccaaccaaaaa

SEQ ID NO: 14 - protein, 145 aa, Homo Sapiens
ARSPLADPPVFLSCWAPNISEITYSWRRETTMDFGMEPHRLFTDGQVLSISLGPGDRDVAYSCIVSNPVSWDLATVTPWDSCHHEAAPGKASYKDVLLVVVPVSLLLMLVTLFSAWHWCPCSGNTILIDILTISIRSHASCRIWE

SEQ ID NO: 15 - DNA, 800 bp, Homo Sapiens
gtagaaagggatgctcagccctccaagacctgccaggttttcttgtcctgttgggcccccaacatcagcgaaataacctatagctggcgacgggagacaaccatggactttggtatggaaccacacagcctcttcacagacggacaggtgctgagcatttccctgggaccaggagacagagatgtggcctattcctgcattgtctccaaccctgtcagctgggacttggccacagtcacgccctgggatagctgtcatcatgaggcagcaccagggaaggcctcctacaaagatgtgctgctggtggtggtgcctgtctcgctgctcctgatgctggttactctcttctctgcctggcactggtgcccctgctcagggccccacctcagatcaaagcagctctggatgagatgggacctgcagctctccctccacaaggtgactcttagcaacctcatttcgacagtggtttgtagcgtggtgcaccagggccttgttgaacagatccacactgctctaataaagttcccatccttaatgactcacttgtcaactagtggactaattaaccctccaccaaaaaaacacaaagtgcttctgtgagaccaattttgtgctaatgagcattgagactgatgctttgtaagtcacaccacaacaaatattgattgagggcgctgcatgtgctgggtacatttcttggcacttgggaatcagtagtcaagcgaaacccttgcctttgagagtttatggtctggataatataaataaacaagtaagcataaaaaaaaaaaaaaaaa

SEQ ID NO: 16 - protein, 200 aa, Homo Sapiens
VERDAQPSKTCQVFLSCWAPNISEITYSWRRETTMDFGMEPHSLFTDGQVLSISLGPGDRDVAYSCIVSNPVSWDLATVTPWDSCHHEAAPGKASYKDVLLVVVPVSLLLMLVTLFSAWHWCPCSGPHLRSKQLWMRWDLQLSLHKVTLSNLISTVVCSVVHQGLVEQIHTALIKFPSLMTHLSTSGLINPPPKKHKVLL

SEQ ID NO: 17 - DNA, 1423 bp, Homo Sapiens
  misc_feature (1)...(1423): n = A,T,C or G
ctaggagcctcctaatgcagtcttctgcacagtcctggggactgatctgactgantcacacctctggggctgggggctgctgacatgtgtgcctttccttggctgcttcttctcctgctgctccaggagggcagccaaaggagactctggagatggtgtggatccgaggaagtggttgcggtccttcaggagtccatcagcctccccctggaaataccaccagatgaagaggttgagaacatcatctggtcctctcacaaaagtcttgccactgtggtgccagggaaagagggacatccagctaccatcatggtgaccaatccacactaccagggccaagtgagcttcctggaccccagctattccctgcatatcagcaatctgagctgggaggattcagggctttaccaagctcaagtcaacctgagaacatcccagatctctaccatgcagcagtacaatctatgtgtctaccgatggctgtcagagccccagatcactgtgaactttgagagttctggggaaggtgcctgcagtatgtccctggtgtgctctgtggagaaggcaggcatggatatgacctacagctggctctcccggggggatagcacttatacattccatgaaggccctgtcctcagcacatcctggaggccgggggacagtgccctctcctacacctgcagagccaacaaccccatcagcaacgtcagttcttgccccatccctgatgggcccttctatgcagatcctaactatgcttctgagaagccttcaacagccttctgcctcctggccaagggattgctcatcttcttgctcttggtaattctggccatgggactctgggtcatccgagtccagaaaagacacaaaatgccaaggatgaagaaactcatgagaaacagaatgaaattgaggaaggaggcaaagcctggctccagccctgcctgactgctccttgggaaccccagtcctgagcttggtttcttcccagcacccagagaatccttcctcagctctcttctttccaggggaaggaggtgctcaggggtgggtatccagagagccatacttctgagggaagactggctggcaataaagtcaaattaagtgaccacaactctgcaggagctgtgttgggtccttccgtcctcactgggtggctctggcaaaacccactctgctgtctttgccccaactcccagtgccttcccccaagtccacgtgctttttcaggccctcctttggggagaagagtgggtctaaagggccccctcacagggaagttttggtatgctgtcactcaccacctatcccgttttccaccaacatgcttgctatttgttcatcagataagaaatgtgagagatttctttgaacctaactgaacctgtgacttgagaatttcaggcattttgaa

SEQ ID NO: 18 - protein, 289 aa, Homo Sapiens
MCAFPWLLLLLLLQEGSQRRLWRWCGSEEVVAVLQESISLPLEIPPDEEVENIIWSSHKSLATVVPGKEGHPATIMVTNPHYQGQVSFLDPSYSLHISNLSWEDSGLYQAQVNLRTSQISTMQQYNLCVYRWLSEPQITVNFESSGEGACSMSLVCSVEKAGMDMTYSWLSRGDSTYTFHEGPVLSTSWRPGDSALSYTCRANNPISNVSSCPIPDGPFYADPNYASEKPSTAFCLLAKGLLIFLLLVILAMGLWVIRVQKRHKMPRMKKLMRNRMKLRKEAKPGSSPA

SEQ ID NO: 19 - DNA, 827 bp, Homo Sapiens
atggcgcacacacctcggtgctctgtacaagagcctcaagaccgagagaaaggactctggtggttccagttgaaggtctggtccatggcagtcgtatccatcttgctcctcagtgtctgtttcactgtgagttctgtggtgcctcacaattttatgtatagcaaaactgtcaagaggctgtccaagttacgagagtatcaacagtatcatccaagcctgacctgcgtcatggaaggaaaggacatagaagattggagctgctgcccaaccccttggacttcatttcagtctagttgctactttatttctactgggatgcaatcttggactaagagtcaaaagaactgttctgtgatgggggctgatctggtggtgatcaacaccagggaagaacaggatttcatcattcagaatctgaaaagaaattcttcttattttctggggctgtcagatccagggggtcggcgacattggcaatgggttgaccagacaccatacaatgaaaatgtcacattctggcactcaggtgaacccaataaccttgatgagcgttgtgcgataataaatttccgttcttcagaagaatggggctggaatgacattcactgtcatgtacctcagaagtcaatttgcaagatgaagaagatctacatataaatgaaatattctccctggaaatgtgtttgggttggcatccaccgttgtagaaagctaaattgattttttaatttatgtgtaagttttgtacaaggaatgcccctaaaatgtttcagcaggctgtcacctattacacttatgatataatccattcaaaaaaaaaaaaaaaa

SEQ ID NO: 20 - protein, 218 aa, Homo Sapiens
MAHTPRCSVQEPQDREKGLWWFQLKVWSMAVVSILLLSVCFTVSSVVPHNFMYSKTVKRLSKLREYQQYHPSLTCVMEGKDIEDWSCCPTPWTSFQSSCYFISTGMQSWTKSQKNCSVMGADLVVINTREEQDFIIQNLKRNSSYFLGLSDPGGRRHWQWVDQTPYNENVTFWHSGEPNNLDERCAIINFRSSEEWGWNDIHCHVPQKSICKMKKIYI

SEQ ID NO: 21 - DNA, 800 bp, Homo Sapiens
ccgccccgcgtccgaagaccgagagaaaggactctggtggttccagttgaaggtctggtccatggcagtcgtatccatcttgctcctcagtgtctgtttcactgtgagttctgtggtgcctcacaattttatgtatagcaaaactgtcaagaggctgtccaagttacgagagtatcaacagtatcatccaagcctgacctgcgtcatggaaggaaaggacatagaagattggagctgctgcccaaccccttggacttcatttcagtctagttgctactttatttctactgggatgcaatcttggactaagagtcaaaagaactgttctgtgatgggggctgatctggtggtgatcaacaccagggaagaacaggatttcatcattcagaatctgaaaagaaattcttcttattttctggggctgtcagatccagggggtcggcgacattggcaatgggttgaccagacaccatacaatgaaaatgtcacattctggcactcaggtgaacccaataaccttgatgagcgttgtgcgataataaatttccgttcttcagaagaatggggctggaatgacattcactgtcatgtacctcagaagtcaatttgcaagatgaagaagatctacatataaatgaaatattctccctggaaatgtgtttgggttggcatccaccgttgtagaaagctaaattgattttttaatttatgtgtaagttttgtacaaaggaatgcccctaaaatgtttcagcaggctgtcacctaatacacttatgatataatccttcaaaaaaaaaaaa

SEQ ID NO: 22 - protein, 211 aa, Homo Sapiens
PRPASEDREKGLWWFQLKVWSMAVVSILLLSVCFTVSSVVPHNFMYSKTVKRLSKLREYQQYHPSLTCVMEGKDIEDWSCCPTPWTSFQSSCYFISTGMQSWTKSQKNCSVMGADLVVINTREEQDFIIQNLKRNSSYFLGLSDPGGRRHWQWVDQTPYNENVTFWHSGEPNNLDERCAIINFRSSEEWGWNDIHCHVPQKSICKMKKIYI

(19) United States
(12) Patent Application Publication
(10) Pub. No.: US 2003/0124575 A1
(43) Pub. Date: Jul. 3, 2003

(54) Organic compounds

(76) Inventors: Gudrun Werner, Wien (AT); William F. Phares, Basel (CH); Markus Jaritz, Wien (AT); Hilmar Lapp, San Diego, CA (US); Frank Stephan Kalthoff, Guntramsdorf (AT)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/220,946

(22) PCT Filed: Mar. 28, 2001
(86) PCT No.: PCT/EP01/03542

(51) Int. Cl. C12Q 1/68; C07H 21/04; A61K 39/395; C12N 9/20; C12P 21/02; C12N 5/06; C07K 16/40

(52) U.S. Cl. 435/6; 435/69.1; 435/198; 435/320.1; 435/325; 536/23.2; 530/388.26; 514/12; 424/146.1

(57) ABSTRACT

Dendritic cell (DC) genes and polypeptide and their function in the identification of compounds which are (ant)agonists; and (ant)agonists to DC polypeptides.

The invention relates to novel genes, e.g. polynucleotides encoding corresponding polypeptides (protein) isolated from dendritic cells. The invention also relates to inhibiting or activating action of such polynucleotides and/or polypeptides (protein).

Dendritic cells (DC) are dedicated antigen presenting leukocytes which play a central role in the induction of primary immune responses and tolerance. In the immature state DC's may reside in different tissues of the body being prepared to capture antigen from invading pathogens. Following antigen capture DC's mature into antigen-presenting cells and migrate into lymphoid organs to activate T cells (Banchereau, M. and R. M. Steinman, Nature 329 [1998] 245-252). For example, Langerhans cells (LC), residing in the skin, have been shown to present a variety of antigens that may be generated in or penetrate into skin. In contact hypersensitivity, topical application of a reactive hapten may activate LC's to migrate out of the epidermis into draining lymph nodes, where LC's may present antigen to selected T-cells. During the contact between LC's and T-cells, LC's may provide signals to the T-cells that induce their proliferation and differentiation into effector cells. Depending on the type of T-cells and the kind of interacting molecules involved cytotoxic, regulatory and helper T-cells may be formed. DC's have also been shown to engulf all kinds of apoptic cells and may therefore play a critical role in the maintenance of tolerance to self-antigens (Steinman, R. M. and K. Inaba, J. Leukoc. Biol. 66 [1999] 205-208). Diseases in which DC's, as the principal regulators of immune responses, play a causal or contributory role may be targets for DC-specific pharmaceutical or iatrigenic intervention, such as in chronic inflammatory diseases, autoimmune diseases, or transplant rejection crisis, and including inflammatory skin diseases such as contact hypersensitivity or atopic dermatitis; and in diseases or syndromes in which a significant pathological component is immune suppression as in, and including, AIDS and cancer.

Dendritic cells may be isolated from peripheral blood by negative selection, i.e. separation from monocytes (CD14+), T-cells (CD3+), B-cells (CD19+) and NK-cells (CD16+) by capturing on specific mAb-coated magnetic beads or panning, e.g. according to a conventional method. Alternatively, dendritic cells may be differentiated in vitro from monocytes isolated from peripheral blood by capturing on anti-CD 14 mAb-coated beads, e.g. according to a conventional method. cDNA libraries of DC's may be generated and gene expression patterns of DC's may be obtained by various hybridisation techniques such as oligonucleotide fingerprinting, substractive hybridisation or RNA profiling, and sequencing, e.g. according to a conventional method.

Genes in isolated DC's have now been found, e.g. the genes set out in TABLE 1, which have the nucleotide sequence set out in TABLE 1, including e.g. allelic variants; and/or splice variants thereof; and/or their complements; and which encode a polypeptide which has the amino acid sequence set out in TABLE 1 or at least 80% identity thereto. Splice variants of a gene are indicated in TABLE 1 by “Splice 1” and “Splice 2”.

TABLE 1

| | Sequence of | |
|---|---|---|
| Gene name | nucleotides | amino acids |
| DCEPR | SEQ ID NO: 1 | SEQ ID NO: 2 |
| DCEPR/SPLICE 1 | SEQ ID NO: 3 | SEQ ID NO: 4 |
| DCTMF | SEQ ID NO: 5 | SEQ ID NO: 6 |
| DCTMF/SPLICE 1 | SEQ ID NO: 7 | SEQ ID NO: 8 |
| DCPLD | SEQ ID NO: 9 | SEQ ID NO: 10 |
| DCIGR | SEQ ID NO: 11 | SEQ ID NO: 12 |
| DCIGR/SPLICE 1 | SEQ ID NO: 13 | SEQ ID NO: 14 |
| DCIGR/SPLICE 2 | SEQ ID NO: 15 | SEQ ID NO: 16 |
| DCLYR | SEQ ID NO: 17 | SEQ ID NO: 18 |
| DCLEC | SEQ ID NO: 19 | SEQ ID NO: 20 |
| DCLEC/SPLICE 1 | SEQ ID NO: 21 | SEQ ID NO: 22 |

The DCEPR gene of SEQ ID NO:1 is related to but not identical to the encephalopsin gene sequence (Blackshaw, S. and S. H. Snyder, J. Neuroscience 19 [1999] 3681-3690, GenBank accession no. AF140242). The DCEPR gene may occur in isolated DC's as a splice variant which consists in part of the nucleotide sequence of SEQ ID NO:3 and which encodes in part a polypeptide which has the amino acid sequence set forth in SEQ ID NO:4 or at least 80% identity thereto. The splice variant of SEQ ID NO:3 is identical to SEQ ID NO:1 but misses nucleotides 429 to 747 which are spliced out at the site GAAAG (splice donor).

The DCTMF gene of SEQ ID NO:5 is a novel member of the tetraspannin (4TM) receptor superfamily (Maecker, H. T., S. C. Todd and S. Levy, FASEB J. 11 [1997] 428-442) and encodes a protein which has the amino acid sequence set forth in SEQ ID NO:6. Its closest relatives are the human CD20 protein (SWISSPROT accession no. Q13963, 26.3% identity in a 213 aa overlap) and the mouse FcεRI β-chain (SWISSPROT accession no. P13386, 31.0% identity in a 200 aa overlap). According to a recent working draft sequence (Genbank accession no. AC018966) the DCTMF gene is located on chromosome 11 which contains also the genes for CD20 and FcεRIβ as well as another member of this family (Adra, C. N. et al., Proc. Natl. Acad. Sci USA 91 [1994] 10178-52). The DCTMF polypeptide of SEQ ID NO:6 contains four typical alphahelical transmembrane domains (aa 52-72, aa 85-105, aa 117-137, aa 186-206), which show the highest degree of conservation to CD20 and FcεRIβ. Both cytoplasmic tails show no homology at all to corresponding domains of other tetraspannins. In addition, a splice variant has been found e.g. in SEQ ID NO:7 (splice site nt 574) in which the exon nt 578-681 of SEQ ID NO:5 is absent. The corresponding protein is shown in SEQ ID NO:8.

The DCPLD gene of SEQ ID NO:9 is a novel member of the phospholipase D family (Liscovitch, M. et al., Biochem J. 345 [2000] 401-415). The corresponding amino acid sequence is shown in SEQ ID NO:10. Its closest relatives are the human HU-K4 protein (SWISSPROT accession no. Q92853, 47.1% identity in a 437 aa overlap) and the mouse Schwannoma-associated protein SAM-9 (SWISSPROT accession no. O35405, 47.9% identity in a 434 aa overlap). According to a recent working draft genomic sequence (Genbank accession no. AC013648) the DCPLD coding region is distributed among 9 exons which are located within 6 kb. Alternative splice products of the DCPLD gene leading to proteins with for instance different N- or C-termini are therefore likely to be formed. The DCPLD protein of SEQ ID NO:10 contains one typical [HxK(x)4D(x)6GSxN, aa 197-214] and one atypical [HxK(x)4E(x)5GxSN] phospholipase D active site motif (Stuckey, J. A. and J. E. Dixon, Nat. Struct. Biol. 6 [1999] 278-284) similar to HU-K4 and SAM-9. The DCPLD polypeptide of SEQ ID NO:10 contains one typical alphahelical transmembrane domain (aa 16-36) and may therefore be associated with the plasma membrane. Through alternative splicing cytoplasmic variants of the DCPLD proteins may exist.

The DCIGR gene of SEQ ID NO:11 encoding the protein set forth in SEQ ID NO:12 is a novel member of the CD2-related immunoglobulin superfamily of type I membrane receptors including the human proteins SLAM (SWISSPROT accession no. Q13291), 2B4 (SWISSPROT accession no. Q9Y288), CD84 (SWISSPROT accession no. O95660, O15430) and CD48 (SWISSPROT accession no. P09326) which show an overall homology between 24% and 27% to DCIGR. This family of proteins includes important co-stimulatory receptors capable of augmenting or perhaps inhibiting antigen-initiated responses (Tangye S. G. et al., Semin. Immunol. 12 [2000] 149-157). Interactions with these co-receptors and their cognate ligands can induce signals resulting in proliferation, cytokine secretion and differention into effector cells. The DCIGR gene of SEQ ID NO:11 shows a long 3′UTR region which has been found to be subject to alternative splicing in dendritic cells. Thereby variants are generated e.g. of SEQ ID NO:13 and of SEQ ID NO:15, encoding polypeptides with different C-terminal domains set forth in SEQ ID NO:14 and set forth in SEQ ID NO:16.

The DCLYR gene of SEQ ID NO:17 encoding the protein of SEQ ID NO:18 is another novel member of the CD2-like family of receptors with two immunoglobulin-like extracellular domains. These domains display the highest degree of homology to LY-9 (TREMBL accession no. AAG14995, 40.2% identity in a 204 amino acid overlap) and to the 19A protein (TREMBL accession no. Q9NY08, 31.7% identity in a 259 amino acid overlap). Similar to DCIGR alternative splicing in the 3′UTR region of DCLYR may lead to isoforms with different cytoplasmic domains. Polymorphism in cytoplasmic domains has been described for several other members of the CD2-like superfamily (Stepp S. E. et al., Eur. J. Immunol. 29 [1999] 2392-9) and may result in the activation of different signaling pathways depending on the interacting adapter molecules (Palou E. et al., Tissue Antigens 55 [2000] 118-127).

The DCLEC gene of SEQ ID NO:19 encoding the polypeptide of SEQ ID NO:20 is related to but not identical to the recently described C-type lectin (Bates E. E. M. et al., J. Immunol. 163 [1999] 1973-1983, SwissProt Accession No. CAB54001). It is a type II transmembrane molecule with a characteristic N-terminal cytoplasmic domain followed by a single transmembrane region (aa 26-46 in SEQ ID NO:20) and an extracellular C-type lectin domain (aa 114-211 in SEQ ID NO:20). In addition, variability in the N-terminal cytoplasmic tail through alternative splicing has been found e.g. in SEQ ID NO:21 (splice site nt 15-20) and the corresponding protein in SEQ ID NO:22.

In one aspect the invention provides an isolated gene which is

a DCEPR gene encoding a polypeptide of SEQ ID NO:2, or of SEQ ID NO:4, or

a DCTMF gene encoding a polypeptide of SEQ ID NO:6, or of SEQ ID NO:8, or

a DCPLD gene encoding a polypeptide of SEQ ID NO:10, or

a DCIGR gene encoding a polypeptide of SEQ ID NO:12, or of SEQ ID NO:14, or of SEQ ID NO:16, or

a DCLYR gene encoding a polypeptide of SEQ ID NO:18, or

a DCLEC gene encoding a polypeptide of SEQ ID NO:20, or of SEQ ID NO:22, or splice variants thereof, including

a DCEPR/SPLICE 1 gene encoding a polypeptide of SEQ ID NO:4, or

a DCTMF/SPLICE 1 gene encoding a polypeptide of SEQ ID NO:8, or

a DCIGR/SPLICE 1 gene encoding a polypeptide of SEQ ID NO:14, or

a DCIGR/SPLICE 2 gene encoding a polypeptide of SEQ ID NO:16, or

a DCLEC/SPLICE 1 gene encoding a polypeptide of SEQ ID NO:22.

In another aspect the invention provides an isolated

DCEPR gene of SEQ ID NO:1, e.g. encoding a polypeptide of SEQ ID NO:2 or of SEQ ID NO:4, or

DCTMF gene of SEQ ID NO:5, e.g. encoding a polypeptide of SEQ ID NO:6, or of SEQ ID NO:8, or

DCPLD gene of SEQ ID NO:9, e.g. encoding a polypeptide of SEQ ID NO:10, or

DCIGR gene of SEQ ID NO:11, e.g. encoding a polypeptide of SEQ ID NO:12, or of SEQ ID NO:14, or of SEQ ID NO:16, or

DCLYR gene SEQ ID NO:17, e.g. encoding a polypeptide of SEQ ID NO:18, or

DCLEC gene of SEQ ID NO:19, e.g. encoding a polypeptide of SEQ ID NO:20, or of SEQ ID NO:22, or splice variants thereof, including

a DCEPR/SPLICE 1 gene of SEQ ID NO:3, e.g. encoding a polypeptide of SEQ ID NO:4, or

a DCTMF/SPLICE 1 gene of SEQ ID NO:7, e.g. encoding a polypeptide of SEQ ID NO:8, or

a DCIGR/SPLICE 1 gene of SEQ ID NO:13, e.g. encoding a polypeptide of SEQ ID NO:14, or

a DCIGR/SPLICE 2 gene of SEQ ID NO:15, e.g. encoding a polypeptide of SEQ ID NO:16, or

a DCLEC/SPLICE 1 gene of SEQ ID NO:21, e.g. encoding a polypeptide of SEQ ID NO:22.

In another aspect the invention provides an isolated polypeptide of SEQ ID NO:2, or of SEQ ID NO:4, or of SEQ ID NO:6, or of SEQ ID NO:8, or of SEQ ID NO:10, or of SEQ ID NO:12, or of SEQ ID NO:14, or of SEQ ID NO:16, or of SEQ ID NO:18, or of SEQ ID NO:20, or of SEQ ID NO:22.

The genes as described above, e.g. a DCEPR, DCTMF, DCPLD, DCIGR, DCLYR and DCLEC-gene, are also designated herein as “gene(s) according to (of) the invention”. Genes according to the invention include a gene of the corresponding sequence as set out in TABLE 1; and allelic variants therof, and their complements; e.g. including a polynucleotide that hybridizes to a nucleotide sequence of a gene according to the invention, e.g. under stringent conditions, e.g. each nucleotide sequence of a gene according to the invention includes a sequence which is different, e.g. as a result of the redundancy (degeneracy) of the genetic code, from the sequence of a gene according to the invention, but also encodes a corresponding polypeptide according to the invention, e.g. of the amino acid sequence as set out in TABLE 1, or encodes e.g. a polypeptide according to the invention of an amino acid sequence which has at least 80% identity with the amino acid sequence of the corresponding polypeptide according to the invention.

Polypeptides as described above, e.g. a DCEPR, DCTMF, DCPLD, DCIGR, DCLYR and DCLEC-polypeptide, are herein also designated as “polypeptide(s) according to (of) the invention”. A polypeptide according to the invention includes a polypeptide of the amino acid sequence as set out in TABLE 1 and includes e.g. an amino acid sequence which has at least 80% identity with the amino acid sequence of the corresponding polypeptide according to the invention as set out in TABLE 1, and e.g. the same biological activity as a polypeptide according to the invention.

“Polypeptide”, if not otherwise specified herein, includes any peptide or protein comprising two or more amino acids joined to each other by peptide bonds.

“Polynucleotide”, if not otherwise specified herein, includes any polyribonucleotide or polydeoxyribonucleotide, which may be unmodified RNA or DNA, or modified RNA or DNA, including without limitation single and double stranded RNA, and RNA that is a mixture of single- and double-stranded regions.

A gene according to the invention includes a polynucleotide comprising the corresponding nucleotide sequence as indicated in TABLE 1; including e.g. allelic variants thereof and/or their complements, and splice variants therof, including e.g. the nucleotide sequences of the corresponding gene indicated in TABLE 1 under “gene name” marked by “/SPLICE 1” or “/SPLICE 2”. A gene according to the invention encodes a polypeptide, or a part of a polypeptide (fragment), according to the invention, e.g. a polypeptide of the corresponding amino acid sequence as set out in TABLE 1, or encodes a polypeptide or a part of a polypeptide of an amino acid sequence which has at least 80% identity with the corresponding amino acid sequence of a polypeptide according to the invention, e.g. as indicated in TABLE 1, e.g. over the entire lenghth of said corresponding amino acid sequence; e.g. 80% to 100%, such as 90%, e.g. 95%, e.g. 97%, e.g. 99% or 100% identity, including a polypeptide encoded by an allelic variant of said gene, or an isofom of the corresponding amino acid sequence generated by alternative splicing of transcripts from the corresponding gene.

“Identity” is a measure of the identity of nucleotide sequences or amino acid sequences and may e.g. be calculated by conventional techniques, using e.g. commercially available computer programs, identity being calculated by the formula

nₐ=xₐ−(Xₐ·y)

wherein nₐ is the number of amino acid alterations, Xₐ is the total number of amino acids in said corresponding amino acid sequence, and y is the percent identity divided by 100.

A gene according to the invention encoding a corresponding polypeptide according to the invention may be obtained using standard cloning and screening methods, e.g. from a cDNA library derived from mRNA of dendritic cells, e.g. using the expressed sequence tag (EST) analysis (Adams, M. D. et al., Science 252 [1991] 1651-1656; Adams, M. D. et al., Nature 355 [1992] 632-634; Adams, M. D. et al., Nature 377 Suppl. [1995] 3-174). A gene acording to invention may also be obtained from natural sources such as genomic DNA libraries or may be synthesized according to a conventional method. The nucleotide sequence of a gene according to the invention encoding a corresponding polypeptide according to the invention may be identical to the corresponding nucleotide sequence of a gene according to the invention, or it may be a sequence which is different, e.g. as a result of the redundancy (degeneracy) of the genetic code, but also encodes a corresponding polypeptide of the invention, having e.g. the same biological activity as a polypeptide according to the invention.

A gene according to the invention may be used for the recombinant production of a corresponding polypeptide (fragment) according to the invention. If a gene according to the invention is used for the recombinant production of a corresponding polypeptide (fragment), the gene sequence may include the coding sequence for the mature polypeptide (fragment) by itself; the coding sequence for the mature polypeptide (fragment) in reading frame with other coding sequences, such as those encoding a leader or secretory sequence, a pre- or pro- or prepro-protein sequence, or other fusion peptide portions. For example, a marker sequence which facilitates purification of a fused polypeptide can be encoded. The marker sequence may be an appropriate marker sequence, e.g. including conventional marker sequences, e.g. a hexa-histidine peptide, as provided in the pQE vector (Qiagen, Inc.) and described in Gentz et al., Proc. Nat. Acad. Sci. USA 86 (1989) 821-824, or an HA tag. Any gene according to the invention may also contain non-coding 5′ and 3′ sequences, such as transcribed, non-translated sequences, splicing and polyadenylation signals, ribosome binding sites and sequences that stabilize mRNA.

A gene according to the invention includes a polynucleotide that hybridizes to the corresponding nucleotide sequence of a gene according to the invention; including e.g. allelic variants thereof and/or their complements or splice variants thereof, e.g. that hybridizes under stringent conditions. “Stringent conditions” includes that hybridization will occur only if there is at least 80%, e.g. 90%, such as 95%, 97% or 99% identity between the nucleotide sequence of a gene according to the invention and the corresponding polynucleotide that hybridizes.

A nucleotide sequence which is identical or sufficiently identical to the nucleotide sequence of a gene according to the invention, e.g. as set out in TABLE 1, e.g. including a fragment thereof or a splice variant therof, e.g. a splice variant as indicated in TABLE 1 by “SPLICE”, may be used as a hybridization probe for cDNA and genomic DNA, to isolate full-length cDNAs and genomic clones encoding a corresponding polypeptide (fragment) according to the invention; and to isolate e.g. cDNA and genomic clones of other genes (including genes encoding homologs and orthologs from species other than human) that have a high sequence similarity to a gene according to the invention.

Hybridization may be carried out e.g. according to a conventional method. Typically a sequence similar to a gene sequence is 80% identical, preferably 90% identical, more preferably 95% identical to that of a gene (fragment) of the invention. A hybridization probe may e.g. comprise at least 15 nucleotides, e.g. at least 30 nucleotides, such as at least 50 nucleotides; e.g. between 30 and 50 nucleotides.

To obtain a polynucleotide encoding a polypeptide according to the invention, including homologs and orthologs from species other than human, any appropriate hybridization technique may be used, e.g. comprising the steps of screening an appropriate library under stringent hybridization conditions with a labeled probe having the corresponding polynucleotide sequence or that of a splice variant thereof or a fragment thereof, and isolating full-length cDNA and genomic clones containing said polynucleotide sequence. Hybridization techniques, e.g. stringent, are well known. Stringent hybridization conditions are e.g. as defined above or, alternatively, conditions under overnight incubation at around 40° C. in an appropriate solution, e.g. comprising a solution comprising formamide, SSC, sodium phosphate, Denhardt's solution, dextran, salmon sperm DNA, e.g. comprising 50% formamide, 5×SSC (150 mM NaCl, 15 mM trisodium citrate), 50 mM sodium phosphate (pH7.6), 5×Denhardt's solution, 10% dextran sulfate, and 20 microgram/ml denatured, sheared salmon sperm DNA, followed by washing the filters in 0×SSC at about 65° C.

In another aspect the invention provides a vector comprising a gene of the invention.

A vector comprising a gene according to the invention may be produced as appropriate, e.g. according to a conventional method, e.g. using an appropriate vector. An appropriate vector may be provided as appropriate, e.g. according to a conventional method. A vector comprising a gene of the invention may be useful to obtain an expression system which is able to produce a polypeptide encoded by a gene according to the invention recombinantly, e.g. in a host cell, such as in a compatible host cell. For e.g. recombinant production of a polypeptide according to the invention a host cell may be genetically engineered, e.g. by use of a vector comprising a gene according to the invention, to incorporate into the host cell an expression system, or a part thereof, for e.g. expressing a polypeptide (fragment) of the invention. Cell-free translation systems may also be used to produce a gene according to the invention, e.g. using RNAs derived from a DNA construct according to the invention, e.g. according to a conventional method.

In another aspect the invention provides an expression system comprising a DNA or RNA molecule isolated from the natural environment, e.g. comprising an pre-isolated gene according to the invention, wherein said expression system or part thereof is capable of producing a corresponding polypeptide, e.g. comprising a polypeptide of the invention as described above, when said expression system or part thereof is present in a compatible host cell.

In another aspect the invention provides:

an isolated host cell comprising an expression system according to the invention;

a process for producing a polypeptide according to the invention comprising culturing an isolated host cell comprising an expression system according to the invention under conditions sufficient for the production of a polypeptide of the invention in the culture and recovering said polypeptide of the invention from the culture;

a process for the production of a recombinant host cell which produces a polypeptide according to the invention comprising transforming or transfecting a host cell with the expression system according to the invention such that the host cell, under appropriate culture conditions, produces a polypeptide according to the invention; and

a recombinant host cell produced by transforming or transfecting a host cell with the expression system according to the invention such that the host cell, under appropriate culture conditions, produces a polypeptide according to the invention.

For recombinant production, host cells may be genetically engineered to incorporate expression systems or portions thereof for a gene according to the invention.

Introduction of polynucleotides into host cells may be effected as appropriate, e.g. according to a conventional method [e.g. according to Davis et al., Basic Methods in Molecular Biology (1986); Sambrook et al., Molecular Cloning: A Laboratory Manual, 2nd Ed., Cold Spring Harbor Laboratory Press, Cold Spring Harbor, N.Y. (1989)], such as calcium phosphate transfection, DEAE-dextran mediated transfection, transvection, microinjection, cationic lipid-mediated transfection, electroporation, transduction, scrape loading, ballistic introduction or infection. Host cells may be easily found. Examples of appropriate host cells include e.g. bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; isolated animal cells such as CHO, COS, HeLa, C127, CCL39, 3T3, BHK, HEK 293 and Bowes melanoma cells; and plant cells.

Appropriate expression systems include e.g. chromosomal, episomal and virus-derived systems, e.g. vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. An expression system may contain control regions that regulate as well as engender expression. Generally, any system or vector suitable to maintain, propagate or express polynucleotides to produce a polypeptide in a host may be used. The appropriate nucleotide sequence may be inserted into an expression system as appropriate, e.g. according to a conventional method, e.g. according to Sambrook et al., Molecular Cloning: A Laboratory Manual (supra).

If a polypeptide according to the invention is to be expressed for use in screening assays, it is generally preferred that the polypeptide is produced at the surface of the cell. In this event, the cells may be harvested prior to use in the screening assay. A polypeptide according to the invention may be recovered and purified from recombinant cell cultures as appropriate, e.g. according to a conventional method including e.g. detergent extraction, ultracentrifugation, ammonium sulfate or ethanol precipitation, acid extraction, anion or cation exchange chromatography, phosphocellulose chromatography, hydrophobic interaction chromatography, affinity chromatography, hydroxylapatite chromatography, lectin chromatography, e.g. high performance liquid chromatography. If a polypeptide according to the invention is denatured during isolation and or purification, regeneration of the active conformation, e.g. refolding of a denaturated polypeptide of the invention, may be carried out as appropriate, e.g. according to a conventional method.

A polypeptide according to the invention includes a polypeptide which is encoded by a corresponding gene according to the invention, including a polynucleotide that hybridizes to the nucleotide sequence of a gene according to the invention; e.g. including a sequence which, as a result of the redundancy (degeneracy) of the genetic code, also encodes a corresponding polypeptide of the invention; or e.g. an allelic variant and/or complement of a gene of the invention.

A polypeptide according to the invention includes a polypeptide with an amino acid sequence according to the invention and with an amino acid sequence which has at least 80% identity with said amino acid sequence. A polypeptide according to the invention may be in the form of the “mature” polypeptide, e.g. protein, or may be part of a larger polypeptide, e.g. protein, e.g. of a fusion protein; it may e.g. be advantageous to include an additional amino acid sequence which contains secretory or leader sequences, pro-sequences, sequences which aid in purification such as multiple histidine residues, or an additional sequence for stability during recombinant production into a polypeptide of the invention.

A polypeptide according to the invention also includes a polypeptide fragment of a polypeptide according to the invention. Such polypeptide fragment is meant to be a polypeptide having an amino acid sequence that entirely is the same in part, but not in all, of the amino acid sequence of a polypeptide of the invention. Such polypeptide fragment may be “free-standing,” or may be part of a larger polypeptide of which such polypeptide fragment forms a part or region, most preferably as a single continuous region. Preferably such polypeptide fragment retains the biological activity of the corresponding polypeptide according to the invention.

Variants of defined polypeptide (fragment) sequences according to the invention also form part of the invention. Preferred variants are those that vary from the referents by conservative amino acid substitutions, e.g. those that substitute a residue with another of like characteristics. Typically such substitutions are among Ala, Val, Leu and lie; among Ser and Thr; among the acidic residues Asp and Glu; among Asn and Gln; and among the basic residues Lys and Arg; or among aromatic residues Phe and Tyr. Particularly preferred are variants in which several, 5 to 10, 1 to 5, or 1 to 2 amino acids are substituted, deleted, or added in any combination.

A polypeptide according to the invention, or e.g. a fragment thereof, includes isolated naturally occurring polypeptides of the invention, or e.g. polypeptide fragments; recombinantly produced polypeptides, or e.g. polypeptide fragments; synthetically produced polypeptides, e.g. polypeptide fragments; or polypeptides, or e.g. polypeptide fragments, produced by a combination of these methods. A polypeptide or fragment thereof of the invention may be produced as appropriate, e.g. according to a conventional method. “Isolated”, if not otherwise specified herein includes the meaning “separated from the coexisting material”, e.g. “altered by the hand of man” from the natural state.

A gene (fragment) according to the invention or a polypeptide (fragment) according to the invention may be used as a research reagent and material for the discovery of treatments and diagnostics to animal and human disease.

A DCEPR gene according to the invention was found to belong to the family of G-protein coupled receptors, typically containing 7 alphahelical transmembrane domains forming a narrow binding pocket for a variety of peptidic and nonpeptidic ligands such as hormones, chemokines, chromophores and neurotransmitters. Agonistic interaction with ligands may result in the binding of a heterotrimeric G-protein to the second intracellular loop of the receptor. Signals to intracellular compartments may be transduced via the second messengers cAMP or IP3 that in turn may induce cascades of signaling events ultimately resulting in a changed phenotype or function of the cell (Ji, T. H., J. Biol. Chem. 273 [1998] 17299-17302; Gether, U. and B. K. Kobilka, J. Biol. Chem. 273 [1998] 17979-17982).

It has now also been found that the DCEPR gene sequence shows characteristic elements of the opsin photoceptors, which are a subfamily of G-protein coupled receptors. A similar protein in the mouse has been shown to be expressed in the brain and testis (Blackshaw, S. and S. H. Snyder, J. Neuroscience 19 [1999] 3681-3690), but not to be expressed in different tissues. The DCEPR gene may thus mediate responses of the dendritic cell type, e.g. the Langerhans cell to light, for instance UV light. Exposure of skin to UV light may result, due to the presence of DCEPR, in immune suppression with particular involvement of the LC—LC's are known to be especially sensitive to UV light and emigrate from skin to draining lymph nodes upon UV exposure, and may initiate events resulting in a reduced systemic immune responsiveness (Kurimoto, I. et al., Immunology 99 [2000] 134-140).

A DCTMF gene according to the invention was found to belong to the superfamily of TM4 receptor molecules, tetraspannins, with CD20 and the Fcε-receptor β-chain as closest relatives. Similar to those molecules DCTMF may associate with antigen receptors, e.g. with the Fcε-receptor α-chain to form a multimeric complex together with the γ-chain, which both are expressed in dendritic cells. In contrast to mast cells and basophils, dendritic cells and monocytes do not express the Fcε-receptor β-chain which has been found to be an important amplifier of signaling events following receptor engagement by antigen and therefore is thought to be critically involved in allergic responses initiated by those cells (J. P. Kinet, Annu. Rev. Immunol. 17 [1999] 943-972). It has been proposed that a β-chain-like molecule, yet to be identified, may substitute for this function in dendritic cells. DCTMF according to the invention, although lacking the typical ITAM motifs present in the β-chain of FcεRI, contains one SH2 domain and two additional tyrosines in its cytoplasmic C-terminal domain and can thus be expected to take part in signal transduction pathways initiated by antigenic activation of dendritic cells. It has now been found that expression of the DCTMF gene according to the invention is indeed regulated in dendritic cells upon activation.

A DCPLD gene according to the invention was found to belong to the phospholipase D family with Hu-K4 (SWISSPROT accession no. Q92853) and SAM-9 (SWISSPROT accession no. O35405) as closest relatives displaying an overall homology of 63% including conservative amino acid changes. DCPLD, Hu-K4 and SAM-9 are distinct members of the PLD family as they do not contain the PX or PH domains of human PLD1 (SWISSPROT accession no. Q13393 or SWISSPROT accession no. O43540). Therefore, their activity may be regulated differentially from the latter ones. DCPLD according to the invention shows the highest degree of conservation to phospholipases D around the phosphatidyltransferase (HKD) motif HxK(x)4D(x)6GSxN which is found in the center of either half of the bilobal catalytic domain. In DCPLD, Hu-K4 and SAM-9, however, the second, C-terminal HKD motif is modified to HxK(x)4E(x)4IGTSN which may indicate a subtle change in the catalytic activity of these enzymes. PLD enzymes have been found to be tightly controlled in their activity in response to extracellular stimuli. They belong to a growing superfamily of phospholipid degrading enzymes including the phosphoinositide-specific enzymes phospholipase A and C as well as sphingomyelinases which all generate biologically active products that are assumed to play important functions in cell regulation (Liscovitch, M. et al., Biochem. J. 345 [2000] 401-415). DCPLD according to the invention contains an alphahelical transmembrane domain in vicinity to its N-terminus and may therefore associate with other signalling transmembrane molecules in dendritic cells and control important steps during differentiation into the professional antigen presenting cell-type.

In another aspect the invention provides an isolated phospholipid degrading enzyme derived from dendritic cells, comprising e.g. a DCPLD polypeptide (protein) according to the invention, e.g. encoded by an DCPLD gene according to the invention.

A DCIGR and a DCLYR gene according to the invention were found to belong to the superfamily of immunoglobulin-like receptors, especially the CD2-like subset including e.g. SLAM (SWISSPROT accession no. Q13291, 26.3% identity in 209 aa overlap) and 2B4 (SWISSPROT accession no. Q9Y288, 24.2% identity in 264 aa overlap). Similar to the other members of this family, DCIGR according to the invention contains one N-terminal V- (variable, immunoglobulin-like) domain followed by a single disulfide-linked immunoglobulin-like C2- (constant) domain, a single alphahelical transmembrane domain (aa 233-253 in SEQ ID NO:12) and variable C-terminal cytoplasmic tails generated through alternative splicing, e.g. as set forth in SEQ ID NO:14 and SEQ ID NO:16. A very similar domain structure has been found for the DCLYR protein set forth in SEQ ID NO:18. Alternative splicing leading to isoforms with different C-terminal cytoplasmic domains has also been observed in the case of murine 2B4 (Stepp S. E. et al., Fur. J. Immunol. 29 [1999] 2392-2399). It may be assumed that the various isoforms recrute different adapter molecules and induce different signal transduction pathways upon receptor engagement. It has now been observed that the different splice-variants of the DCIGR gene according to the invention are expressed at varying levels in dendritic cells that are activated by different stimuli, e.g. LPS-treatment leading to maturation of DC or IL-10 treatment inducing a toleragenizing state. Furthermore, it was found that DCIGR according to the invention is also expressed in T-cells and monocytes, although to a lesser degree, and regulated differently from DC during activation of those cell-types. Thus, DCIGR isoforms may play an important role in signal transduction in dendritic cells, T-cells and monocytes and therefore, binding of agonists or antagonists to DCIGR according to the invention may alter the biological response following antigenic activation of these cells.

A DCLEC gene according to the invention was found to belong to the family of C-type lectins with one single carbohydrate recognition domain at the COOH-terminal end. As a type II transmembrane receptor it typically contains one alphahelical transmembrane domain and a N-terminal cytoplasmic tail. Other family members include DCIR expressed in dendritic cells, as well as a number of genes located in the NK gene complex, NKR-P1, Ly-49, NKG2, CD94, CD69, AICL and LLTR (Boles K. S, Immunogenetics 50 [1999] 1-7). While most of these receptors contain ITIM motifs in their cytoplasmic domain and are involved in negative signalling, no such motif has been identified in DCLEC, but it is not excluded since SEQ ID NO:21 does not represent the complete cDNA and other splice variants may exist. C-type lectins bind oligosaccharide groups and may be involved, similar to asialo-glycoprotein receptors of macrophages, in antigen internalization and uptake of dendritic cells and thus constitute an important first step in antigen presentation (Bates E. E. M. et al., J. Immunol. 163 [1999] 1973-1983). DCLEC may also play an important role in signal transduction in dendritic cells and therefore, binding of agonists or antagonists to DCLEC may alter the biological response during differentiation into antigen presenting.

The present invention also provides the use of a gene according to the invention as a diagnostic reagent. Detection of a mutated form of a gene according to the invention associated with a dysfunction will provide a diagnostic tool, e.g. in a diagnostic assay, that may add to or define a diagnosis of a disease or susceptibility to a disease which results from under-expression, over-expression or altered expression of the corresponding gene or a mutant version thereof. Individuals carrying mutations in the corresponding gene may be detected at the DNA level according to a conventional method. Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in the analysis similarly. Deletions and insertions may be detected by a change in size of the amplified product in comparison to the normal genotype. Point mutations may be identified by hybridizing amplified DNA to labeled gene nucleotide sequences of the invention. Perfectly matched sequences may be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing, e.g. according to Myers et al., Science 230 (1985) 1242. Sequence changes at specific locations may also be revealed by nuclease protection assays, such as RNase and S1 protection or the chemical cleavage method, e.g. according to Cotton et al., Proc. Natl. Acad. Sci. USA 85 (1985) 4397-4401. An array of oligonucleotide probes comprising the gene nucleotide sequence of the invention or fragments thereof may be constructed to conduct efficient screening of e.g. genetic mutations. Array technology methods may e.g. be used to address a variety of questions in molecular genetics including gene expression, genetic linkage, and genetic variability, e.g. according to M. Chee et al., Science 274 (1996) 610-613.

A diagnostic assay offers a process for diagnosing or determining a susceptibility to diseases, e.g. chronic inflammatory diseases, autoimmune diseases, transplant rejection crisis, including e.g. inflammatory skin diseases such as contact hypersensitivity, atopic dermatitis, or virally induced immune suppression, e.g. AIDS, through detection of mutation in the gene according to the invention as appropriate, e.g. including a conventional method, or according to a method as described herein. In addition, diseases such as chronic inflammatory diseases, autoimmune diseases or transplant rejection crisis, including inflammatory skin diseases such as contact hypersensitivity or atopic dermatitis; and diseases or syndromes in which a significant pathological component is immune suppression, as in, and including, AIDS and cancer, may be diagnosed e.g. according to a conventional method, e.g. comprising determining from a sample derived from a subject an abnormally decreased or increased level of a polypeptide according to the invention or of a gene mRNA according to the invention. Decreased or increased expression can be determined at the RNA level e.g. according to a conventional method for the quantitation of polynucleotides, such as, for example, PCR, RT-PCR, RNase protection, Northern blotting and other hybridization methods. Assay techniques that may be used to determine levels of a protein, such as a polypeptide according to the invention, in a sample derived from a host may be carried out e.g. according to a conventional method. Such assay techniques include radioimmunoassays, competitive-binding assays, Western Blot analysis and ELISA assays.

Thus in another aspect, the invention provides a diagnostic kit for a disease or susceptibility to a disease, such as chronic inflammatory diseases, autoimmune diseases, or transplant rejection crisis, including inflammatory skin diseases such as contact hypersensitivity or atopic dermatitis; and in diseases or syndromes in which a significant pathological component is immune suppression, as in, and including, AIDS and cancer, comprising as a main component

a) a gene according to the invention, e.g. including allelic variants thereof, or a fragment thereof; or a splice variant thereof, or

b) a nucleotide sequence complementary to that of (a), or

c) a polypeptide according to the invention, e.g. including a polypeptide of an amino acid sequence which has at least 80% identity thereto, e.g. including a fragment or a variant of a polypeptide according to the invention or a fragment or a variant of a polypeptide of an amino acid sequence which has at least 80% identity to said polypeptide of the invention, or

d) an antibody to a polypeptide according to the invention.

Any such kit (a), (b), (c) or (d) may comprise a substantial component, including e.g. an appropriate environment of a sample to be tested, and appropriate means to determine the effect of any of a), b), c) or d) in a sample to be tested.

A gene according to the invention may also be useful for chromosome identification. The sequence is specifically targeted to and can hybridize with a particular location on an individual human chromosome. The mapping of relevant sequences to chromosomes according to the invention is an important first step in correlating those sequences with gene associated disease. Once a sequence has been mapped to a precise chromosomal location, the physical position of the sequence on the chromosome may be correlated with genetic map data. Corresponding data is disclosed uin e.g. V. McKusick, Mendelian Inheritance in Man (available on-line through Johns Hopkins University Welch Medical Library). The relationship between genes and diseases that have been mapped to the same chromosomal region may be identified through linkage analysis (coinheritance of physically adjacent genes). The differences in the cDNA or genomic sequence between affected and unaffected individuals may also be determined. If a mutation is observed in some or all of the affected individuals but not in any normal individuals, then the mutation is likely to be the causative agent of the disease.

A polypeptide according to the invention or fragment thereof, or cells expressing a polypetide according to the invention can also be used as immunogens to produce antibodies immunospecific for said polypeptide of the invention. The term “immunospecific” means that the antibodies have substantially greater affinity for the said polypeptide of the invention than their affinity for other related polypeptides. Antibodies generated against a polypeptide according to the invention may e.g. be obtained by administering the polypeptide or an epitope-bearing fragment-analogue or cell to an animal, preferably a non-human, using routine protocols. For preparation of monoclonal antibodies, an appropriate technique which provides antibodies, e.g. produced by continuous cell line cultures, may be used, e.g. including the hybridoma technique (Kohler, G. and C. Milstein, Nature 256 [1975] 495-497), the trioma technique, the human B-cell hybridoma technique (Kozbor et al., Immunology Today 4 [1983] 72) and the EBV-hybridoma technique (Cole et al., Monoclonal Antibodies and Cancer Therapy [1985] 77-96, Alan R. Liss, Inc.). Techniques for the production of single chain antibodies (see e.g. U.S. Pat. No. 4,946,778) can also be adapted to produce single chain antibodies to a polypeptide according to the invention. Also, transgenic mice or other organisms, including other mammals, may be used to express humanized antibodies. Antibodies as described above may be used e.g. in the isolation or in the identification of a clone expressing a polypeptide according to the invention or for the purification of a polypeptide (fragment) according to the invention by affinity chromatography. Antibodies against a polypeptide according to the invention may also be useful in the treatment of diseases, e.g. chronic inflammatory diseases, autoimmune diseases, transplant rejection crisis, including e.g. inflammatory skin diseases such as contact hypersensitivity, atopic dermatitis, or in virally induced immune suppression, e.g. AIDS.

In another aspect the invention thus provides an antibody against a polypeptide of the invention.

In another aspect the invention provides a method for inducing an immunological response in a mammal which comprises inoculating the mammal with a polypeptide of the invention, or a fragment thereof, adequate to produce antibody and/or T-cell immune response to protect said animal from diseases, such as in e.g. chronic inflammatory diseases, autoimmune diseases, or transplant rejection crisis, including inflammatory skin diseases such as contact hypersensitivity or atopic dermatitis; and in diseases or syndromes in which a significant pathological component is immune suppression as in, and including, AIDS and cancer.

In another aspect the invention provides a method of inducing an immunological response in a mammal which comprises delivering a polypeptide according to the invention via a vector directing expression of a corresponding gene according to the invention in vivo in order to induce such an immunological response to produce antibodies to protect said animal from diseases; and in another aspect, an immunological/vaccine formulation (composition) which, when introduced into a ma host, induces an immunological response in that mammal to a polypeptide according to the invention, wherein the composition comprises a polypeptide according to the invention or a gene according to the invention.

A vaccine formulation may further comprise a suitable carrier. Since a polypeptide according to the invention may be broken down in the stomach, it is preferably administered parenterally (including subcutaneous, intramuscular, intravenous, intradermal, etc. injection). Immunological/vaccine formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other appropriate systems known in the art. The dosage will depend on the specific activity of the vaccine and can be readily determined by routine experimentation.

A polypeptide according to the invention may be responsible for many biological functions, including those underlying many pathological states. Accordingly, it is desirable to find compounds and drugs which stimulate a polypeptide according to the invention or expression of a gene according to the invention on the one hand (agonists), or which can inhibit the function of a polypeptide according to the invention or expression of a gene according to the invention on the other hand (antagonists). A polypeptide according to the invention or functional mimetics thereof, e.g. according to Coligan et al., Current Protocols in Immunology 1(2) (1991) Chapter 5, may thus be used to assess the binding of agonists or antagonists of the receptor polypeptide of the invention, e.g. in cells, cell-free preparations, chemical libraries, and natural product mixtures, e.g. in a screening assay. Agonists and antagonists of a polypeptide according to the invention may be used in the treatment of diseases, such as in chronic inflammatory diseases, autoimmune diseases, or transplant rejection crisis, and including inflammatory skin diseases such as contact hypersensitivity or atopic dermatitis; and in diseases or syndromes in which a significant pathological component is immune suppression as in, and including, AIDS and cancer. Stimulation or inhibition of expression of a gene according to the invention, e.g. by low molecular weight (LMW) compounds or antisense oligonucleotides, may be desirable to modulate the effects of a corresponding polypeptide according to the invention and its ligands on the physiology/function of the DC.

Screening procedures may involve the production of appropriate cells which express receptors of a polypeptide sequence according to the invention, e.g. on the cell surface. Appropriate cells include cells from e.g. mammals, yeast and Drosophila. Cells expressing the receptor (or cell membranes containing the expressed receptor) may be contacted with a test compound to observe binding, or stimulation or inhibition of a functional response. A screening assay may be used to test binding of a candidate compound wherein adherence to the cells bearing the receptor may be detected by means of a label directly or indirectly associated with the candidate compound or in an assay involving competition with a labeled competitor. A screening assay may be used further to test whether the candidate compound results in a signal generated by activation of the receptor, using detection systems appropriate to the cells bearing the receptor at their surfaces. Inhibitors of activation may be assayed in the presence of a known agonist and the effect on activation by the agonist by the presence of the candidate compound is observed.

A screening assay may comprise the steps of mixing a candidate compound with a solution containing a polypeptide according to the invention to form a mixture, determining activity of said polypeptide in the mixture, and comparing the activity of the mixture with the activity of a standard. A gene (cDNA) according to the invention, a polypeptide according to the invention and antibodies to a polypeptide according to the invention may also be used to provide a screening assay for detecting the effect of candidate compounds on the production of said gene (mRNA) and said polypeptide in cells. For example, an ELISA may be constructed for determining cell associated levels of said polypeptide, e.g. using monoclonal and polyclonal antibodies according to a method as conventional, and that ELISA may be used to discover agents (agonists or antagonists) which may inhibit or enhance the production or the activity of said polypeptide (antagonist, or agonist) in suitably manipulated cells or tissues. An assay for screening may be conducted according to a conventional method.

Examples of potential (ant)agonists of a gene according to the invention include antibodies or, in some cases, oligonucleotides or proteins (polypeptides) closely related to the ligand (antagonist bound to a polypeptide of said gene) of said gene, e.g. a fragment of said ligand, or small molecules, which bind to the receptor but do not elicit a response, so that the activity of the receptor is prevented. Examples of potential (ant)agonists include compounds which bind to a polypeptide according to the invention, e.g. including oligopeptides, polypeptides, protein, antibodies, mimetics, small molecules, e.g. low molecular weight (LMW) compounds.

Thus in another aspect, the invention provides a screening assay for identifying an agonist or an antagonist of a polypeptide according to the invention which assay comprises as a main component

a) a polypeptide according to the invention, or

b) a recombinant cell expressing a polypeptide according to the invention, or

c) a cell membrane expressing a polypeptide according to the invention, or

d) an antibody to a polypeptide according to the invention;

and e.g. means for a contact with a candidate compound; and e.g. means for determining the effect of the candidate compound on any of a), b), c) or d), e.g. determining whether in the presence of the candidate compound there is a decrease or enhancement in the production and/or the biological activity of a polypeptide according to the invention; e.g. by comparison of the activity of any of a), b), c) or d) in the presence and in the absence of the candidate compound;

and in another aspect,

a method of identifying an agonist or antagonist of a polypeptide of the invention, preferably agonist, including e.g. ligands, receptors, antibodies or LMW compounds, which decreases or enhances the production and/or the biological activity of a polypeptide according to the invention, which method comprises

A) contacting

a) a polypeptide according to the invention, or

b) a recombinant cell expressing a polypeptide according to the invention, or

c) a cell membrane expressing a polypeptide according to the invention, or

d) an antibody to a polypeptide according to the invention with a candidate compound,

B) determining the effect of the candidate compound on any of a), b), c) or d);

e.g. by determining whether in the presence of the candidate compound there is a decrease or enhancement in the production and/or the biological activity of a polypeptide according to the invention; e.g. by comparison of the activity of any of a), b), c) or d) in the presence and in the absence of the candidate compound; and

C) choosing an agonist or antagonist determined in step B), e.g., choosing an appropriate candidate compound from which an agonist/antagonistic effect is positively determined in step B).

It will be appreciated that in any such screening assay, a), b), c) or d) may comprise a substantial component. A candidate compound includes compound (libraries) from which the effect on any of a), b), c) or d) is unknown. Compound (libraries) include compounds which are set out above as (ant)agonists to a polypeptide according to the invention. An (ant)agonist is a candidate compound from which an effect on any of a), b), c) or d) has been found in a screening assay or in a method for identifying (ant)agonists as described above. An (ant)agonist may decrease or enhance the production and or the biological activity of a polypeptide according to the invention.

In another aspect the invention provides an antagonist or an agonist, preferably an antagonist, of a polypeptide according to the invention, which is characterized in that said antagonist or agonist can be provided by the following method steps:

A) contacting

a) a polypeptide according to the invention, or

b) a recombinant cell expressing a polypeptide according to the invention, or

c) a cell membrane expressing a polypeptide according to the invention, or

d) an antibody to a polypeptide according to the invention with a candidate compound,

B) determining the effect of the candidate compound on any of a), b), c) or d);

e.g., determining whether in the presence of the candidate compound there is a decrease or enhancement in the production and/or the biological activity of a polypeptide according to the invention; e.g., by comparison of the activity of any of a), b), c) or d) in the presence and in the absence of the candidate compound; and

C) choosing an agonist or antagonist determined in step B); e.g., choosing an appropriate candidate compound from which an agonist/antagonist effect is positively determined in step B).

An (ant)agonist of a polypeptide according to the invention may have immune modulatory activities and may be used in the treatment of diseases, such as in chronic inflammatory diseases, autoimmune diseases, or transplant rejection crisis, including inflammatory skin diseases such as contact hypersensitivity or atopic dermatitis; and in diseases or syndromes in which a significant pathological component is immune suppression as in, and including, AIDS and cancer. An (ant)agonist of a polypeptide according to the invention may thus be useful as a pharmaceutical.

For that use several approaches are available:

If the activity of a gene and/or polypeptide according to the invention is in excess, one approach comprises administering to a subject an antagonist of a polypeptide according to the invention, e.g. in combination with a pharmaceutically acceptable excipient, in an amount effective to inhibit activation of a gene and/or polypeptide according to the invention by blocking binding of ligands to said polypeptide, or by inhibiting a second signal, and thereby alleviating the abnormal condition caused by e.g. over-, under-, or altered expression of said gene (or a mutant version therof). In another approach, soluble forms of a corresponding polypeptide according to the invention, still capable of binding the ligand in competition with endogenous polypeptide, may be administered. Typical embodiments of such competitors may comprise fragments of said polypeptide of the invention. In still another approach, expression of the gene encoding endogenous polypeptide according to the invention may be inhibited using expression blocking techniques. Known such techniques involve the use of antisense sequences, either internally generated or separately administered, e.g. according to O'Connor, J. Neurochem 56 (1991) 560, in Oligodeoxynucleotides as Antisense Inhibitors of Gene Expression, CRC Press, Boca Raton, Fla., USA (1988). Alternatively, oligomers, e.g. oligonucleotides which form triple helices with a gene according to the invention may be supplied, e.g. according to Lee et al., Nucleic Acids Res. 6 (1979) 3073; Cooney et al., Science 241 (1988) 456; Dervan et al., Science 251 (1991) 1360. Such oligomers may be administered per se or may be expressed in vivo.

For treating abnormal conditions related with an under-expression of a polypeptide according to the invention and its activity, one approach comprises administering to a subject in need of an increased expression of a polypeptide according to the invention a therapeutically effective amount of a compound which activates a gene according to the invention (agonist), e.g. in combination with a pharmaceutically acceptable excipient, to thereby alleviate the abnormal condition. Alternatively, gene therapy may be employed to effect the endogenous production of a polypeptide according to the invention by the relevant cells in the subject. For example, a gene according to the invention may be engineered for expression in a replication defective retroviral vector, e.g. according to a method as discussed above. A retroviral expression construct obtained may be isolated and introduced into a packaging cell transduced with a retroviral plasmid vector containing RNA encoding a polypeptide corresponding to said gene according to the invention such that the packaging cell produces infectious viral particles containing the gene of interest. These producer cells may be administered to a subject for engineering cells in vivo and expression of the polypeptide in vivo. For overview of gene therapy, see e.g. Chapter 20, Gene Therapy and other Molecular Genetic-based Therapeutic Approaches (and references cited therein) in Human Molecular Genetics, T. Strachan and A. P. Read, BIOS Scientific Publishers Ltd (1996).

In another aspect the invention thus provides an antagonist or an agonist, preferably an antagonist, of a polypeptide according to the invention for use as a pharmaceutical, e.g. in the treatment of diseases such as in chronic inflammatory diseases, autoimmune diseases, or transplant rejection crisis, including inflammatory skin diseases such as contact hypersensitivity or atopic dermatitis; and in diseases or syndromes in which a significant pathological component is immune suppression as in, and including, AIDS and cancer; and, in another aspect, a soluble form of a polypeptide according to the invention for use as a pharmaceutical, e.g. for the treatment of the same diseases wherein an (ant)agonist of the invention is suitable.

An (ant)antagonist of a polypeptide according to the invention may be administered in the form of a pharmaceutical composition. In another aspect the invention provides a pharmaceutical composition comprising an agonist or an antagonist, preferably an antagonist of a polypeptide according to the invention as an active ingredient in combination with pharmaceutically acceptable excipients/carriers. Said antagonist or agonist can be provided e.g. by the following method steps:

A) contacting

a) a polypeptide according to the invention, or

b) a recombinant cell expressing a polypeptide according to the invention, or

c) a cell membrane expressing a polypeptide according to the invention, or

d) an antibody to a polypeptide according to the invention with a candidate compound,

B) determining the effect of said candidate compound on any of a), b), c) or d);

e.g., determining whether in the presence of the candidate compound there is a decrease or enhancement in the production and or the biological activity of a polypeptide according to the invention; e.g. by comparison of the activity of any of a), b), c) or d) in the presence and in the absence of the candidate compound;

C) choosing an agonist or antagonist determined in step B), e.g., choosing an appropriate candidate compound from which an agonist/antagonist effect is positively determined in step B).

In another aspect, the invention provides a pharmaceutical composition comprising a soluble form of a polypeptide according to the invention as an active ingredient in combination with pharmaceutically acceptable excipient(s)/carriers.

Such pharmaceutical composition may be produced as appropriate, in conventional manner, e.g. by mixing an (ant)agonist provided by the above method steps A), B) and C) with excipients, and further processing the mixture obtained to obtain a pharmaceutical composition for appropriate administration.

In a further aspect the invention provides a method of treating abnormal conditions related to both an excess of and insufficient level, preferably an excess, of expression of a gene according to the invention; or related to both an excess and insufficient activity of a polypeptide according to the invention, preferably an excess; e.g. a method of treating diseases, such as chronic inflammatory diseases, autoimmune diseases, or transplant rejection crisis, including inflammatory skin diseases such as contact hypersensitivity or atopic dermatitis; and diseases or syndromes in which a significant pathological component is immune suppression as in, and including, AIDS and cancer; comprising administering a therapeutically effective amount of an agonist or antagonist to a polypeptide according to the invention, e.g. which can be provided by the method steps A), B) or C) as described above, e.g. in combination with pharmaceutically acceptable excipient(s); or administering a therapeutically effective amount of a soluble form of a polypeptide according to the invention, e.g. in combination with pharmaceutically acceptable excipients; to a subject in need of said treatment.

Preferred forms of systemic administration of a pharmaceutical composition according to the invention include injection, typically by intravenous injection. Other injection routes, such as subcutaneous, intramuscular, or intraperitoneal, may be used. Alternative means for systemic administration include transmucosal and transdermal administration, e.g. using penetrants such as bile salts or fusidic acids or other detergents. In addition, if properly formulated in enteric or encapsulated formulations, oral administration may also be possible. Administration of a composition according to the invention may also be topical and/or localized, e.g. in the form of creams, pastes, gels and the like.

The dosage range required may depend upon the choice of the polypeptide according to the invention, or on the choice of an (ant)agonist of a polypeptide of the invention, the route of administration, the nature of the pharmaceutical composition, the nature of the subject's condition, and the judgment of the attending practitioner. Suitable dosages, however, may be in the range of 0.1 to 1000 μg/kg of subject, e.g. 0.1 to 100 μg/kg of subject. Variations in the needed dosage, however, may be expected in view of the variety of compounds available and the differing efficiencies of various routes of administration. For example, oral administration would be expected to require higher dosages than administration by intravenous injection. Variations in these dosage levels may be adjusted as appropriate, e.g. according to standard empirical routine for optimization.

Polypeptides used in treatment may also be generated endogenously in a subject in need of such treatment, in treatment modalities often referred to as “gene therapy”, e.g. as described above. Thus, for example, cells from a subject may be engineered ex vivo with a polynucleotide, such as a DNA or RNA, to encode a polypeptide according to the invention, e.g. by use of a retroviral plasmid vector. Engineered cells may be introduced into the subject in need of such treatment.

The following abbreviations are used herein:

| | |
|---|---|
| aa = | amino acid |
| DC = | dendritic cell |
| LC = | Langerhaus cell |
| mAb = | monoclonal antibody |
| nt = | nucleotide |

In the following Examples all temperatures are in degree Celsius. A

DCEPR gene (DCEPR/SPLICE 1 is a splice variant thereof) or DCEPR polypeptide,

DCTMF gene (DCTMF/SPLICE 1 is a splice variant thereof), or DCTMF polypeptide,

DCPLD gene, or DCPLD polypeptide,

DCIGR gene (DCIGR/SPLICE 1 and DCIGR/SPLICE 2 are splice variants thereof), or DCIGR polypeptide,

DCLYR gene, or DCLYR polypeptide, and

DCLEC gene (DCLEC/SPLICE 1 is a splice variant thereof), or DCLEC polypeptide, mentioned in the Examples, has the corresponding sequence set out in TABLE 1.

### EXAMPLE 1

### Homology

cDNA clones comprising SEQ ID NO:1 and SEQ ID NO:3 of a DCEPR gene have been isolated from libraries of dendritic cell mRNA and by sequence comparison using the BLASTX algorithm with the SWISSPROT Protein database found to be homologous to the family of seven transmembrane spanning G-protein coupled receptors, especially the opsin subfamily, with encephalopsin (Accession No. Q9Y344) being the closest homolog. The DCEPR gene of SEQ ID NO:1 shows an overall homology to encephalopsin of 98.2%, but with characteristic differences, including a short deletion in the region 1123 to 1209 encoding part of the cytoplasmic tail of the DCEPR polypeptide (SEQ ID NO:2, amino acids 375 to 402). This domain of opsin receptors is critically involved in receptor desensitization (Schertler, G. F., Eye 12 [1998] 504-510) and may thus contribute to DC specific signalling events of the DCEPR polypeptide.

cDNA clones comprising SEQ ID NO:5 and SEQ ID NO:7 of a DCTMF gene have been isolated from libraries of dendritic cell mRNA and by sequence comparison using the BLASTX algorithm found to be homologous to the family of tetraspannins, transmembrane receptors with four alphahelical transmembrane domains. In these domains DCTMF shows the highest degree of homology to known members of the TM4 family, especially to the human CD20 protein (SWISSPROT accession no. Q13963, 26.3% identity in 213 aa overlap) and the mouse FcεRI β-chain (SWISSPROT accession no. P13386, 31.0% identity in 200 aa overlap).

cDNA clones comprising SEQ ID NO:9 of a DCPLD gene have been isolated from libraries of dendritic cell mRNA and by sequence comparison using the BLASTX algorithm found to be homologous to the family of phospholipases D, enzymes that catalyse the hydrolysis of phospholipids such as phosphatidylcholin, phosphatidylinositol or phosphatidayethanolamine. DCPLD shows the highest degree of homology to family members which are as yet not functionally well characterised, namely to the human HU-K4 protein (SWISSPROT accession no. Q92853, 47.1% identity in 437 aa overlap) and the mouse Schwannoma-associated protein SAM-9 (SWISSPROT accession no. 035405, 47.9% identity in 434 aa overlap).

cDNA clones comprising SEQ ID NO:11 and SEQ ID NO:13 and SEQ ID NO:15 of a DCIGR gene have been isolated from libraries of dendritic cell mRNA and by sequence comparison using the BLASTX algorithm found to be homologous to immunoglobulin-like receptors, with SLAM (SWISSPROT accession no. Q13291, 26.3% identity in 209 aa overlap), and 2B4 (SWISSPROT accession no. Q9Y288, 24.2% identity in 264 aa overlap) being the closest human homologs. The highest degree of homology is found in the second C2-immunoglobulin-like extracellular domain, whereas the cytoplasmic tails of all 3 DCIGR isoforms are different from other family members. Additional splice-variants generating different V-domains at the N-terminus also seem to exist, as indicated in SEQ ID NO:13 and the corresponding polynucleotide in SEQ ID NO:14.

cDNA clones comprising SEQ ID NO:17 of a DCLYR gene have been isolated from libraries of dendritic cell mRNA and by sequence comparison using the BLASTX algorithm found to be homologous to the family of immunoglobulin-like receptors, with LY-9 (TREMBL accession no. AAG14995, 40.2% identity in a 204 amino acid overlap) and the 19A protein (TREMBL accession no. Q9NY08, 31.7% identity in a 259 amino acid overlap) being the closest human homologs.

cDNA clones comprising SEQ ID NO:19 and SEQ ID NO:21 of a DCLEC gene have been isolated from libraries of dendritic cell mRNA and by sequence comparison using the BLASTX algorithm found to be homologous to the family C-type lectins, with DCIR (TREMBL accession no. CAB54001, 50.8% identity in 191 aa overlap) being the closest human homolog, and dectin 2-alpha (SWISSPROT accession no. AAF67177, 51% identity in 201 aa overlap) being the most closely related mouse protein. The homology to the C-type lectins does not include the N-terminal cytoplasmic domain which is important in intracellular signalling.

### EXAMPLE 2

### Sequencing

The complete cDNA of an DCEPR, DCTMF, DCPLD, DCIGR, DCLYR and DCLEC gene may be obtained by either of the following methods: a) The method of Rapid Amplification of cDNA Ends (RACE) can be utilized to obtain the 5′-end (see Frohman et al., Proc. Nat. Acad. Sci USA 85 [1988] 8998-9002). Briefly, specific oligonucleotides are annealed to mRNA and used to prime the synthesis of the cDNA strand. Following destruction of the mRNA with RNaseH, a poly C anchor sequence is added to the 3′-end of the cDNA and the resulting fragment is amplified using a nested set of antisense primers and an anchor sequence primer. The amplified fragment is cloned into an appropriate vector and subjected to restriction and sequence analysis. b) The polymerase chain reaction can be used to amplify the 5′ end of the cDNA from human cDNA libraries using sequential rounds of nested PCR with two sets of primers. One set of antisense primers is specific to the 5′ end of the partial cDNA and the other set of primers anneals to a vector specific sequence. The amplified products are cloned into an appropriate vector and subjected to restriction and sequence analysis.

The corresponding sequences of the genes set out in TABLE 1 are obtained.

### EXAMPLE 3

### Expression in Mammalian Cells

The receptors of the DCEPR, DCTMF DCIGR, DCLYR and DCLEC genes of the invention are expressed in either human embryonic kidney 293 (HEK293) cells or adherent CCL39 or dhfr CHO cells and the DCPLD gene of the invention is expressed in either human embryonic kidney 293 (HEK293) cells or adherent CCL39 or dhfr CHO cells or recombinant baculovirus-infected Sf9 cells. The expression vectors typically contain the coding regions free of 5′ and 3′ UTR's downstream of a strong promoter, e.g. CMV-IE and a Koscak sequence as well as an antibiotic resistance gene, e.g. neomycin or zeocin. The cells are transfected with individual receptor cDNAs by lipofectin and selected in the presence of 600 to 1000 mg/ml of G418 (neomycin) or 100 to 400 mg/ml of zeocin. After 3 weeks of selection, individual clones are picked and expanded for further analysis. HEK293 or CHO cells transfected with the vector alone serve as negative controls. To isolate cell lines stably expressing the individual receptors, about 96 clones are typically selected and analyzed by RT PCR analysis. Receptor mRNAs are generally detectable in about 50% of the G418-resistant clones analyzed.

Recombinant baculoviruses for expression of PLD are generated, selected, purified and propagated using standard techniques.

### EXAMPLE 4

### Ligand Screening with DCEPR

A collection of putative receptor ligands has been assembled for screening. The collection comprises: transmitters, hormones and chemokines known to act via a human seven transmembrane (7TM) receptor; naturally occurring compounds which may be putative agonists for a human 7TM receptor, non-mammalian, biologically active peptides for which a mammalian counterpart has not yet been identified; and compounds not found in nature, but which activate 7TM receptors with unknown natural ligands. This collection is used to initially screen the receptor for known ligands, using both functional (i.e. calcium, cAMP, microphysiometer, oocyte electrophysiology, etc; see below) as well as binding assays.

### EXAMPLE 5

### Ligand Binding Assays

Ligand binding assays provide a direct method for ascertaining receptor pharmacology and are adaptable to a high throughput format. The purified ligand for a receptor is radiolabeled to high specific activity (50-2000 Ci/mmol) for binding studies. A determination is then made that the process of radio labeling does not diminish the activity of the ligand towards its receptor. Assay conditions for buffers, ions, pH and other modulators such as nucleotides are optimized to establish a workable signal to noise ratio for both membrane and whole cell receptor sources. For these assays, specific receptor binding is defined as total associated radioactivity minus the radioactivity measured in the presence of an excess of unlabeled competing ligand. Where possible, more than one competing ligand is used to define residual nonspecific binding.

### EXAMPLE 6

### Functional Assay in Xenopus Oocytes

Capped RNA transcripts from linearized plasmid templates encoding the receptor cDNAs of genes of the invention are synthesized in vitro with RNA polymerase in accordance with standard procedures. In vitro transcripts are suspended in water at a final concentration of 0.2 mg/ml. Ovarian lobes are removed from adult female toads, Stage V defolliculated oocytes are obtained, and RNA transcripts (10 ng/oocyte) are injected in a 50 nl bolus using a microinjection apparatus. Two electrode voltage clamps are used to measure the currents from individual Xenopus oocytes in response to agonist exposure. Recordings are made in Ca²⁺ free Barth's medium at room temperature. The Xenopus system can be used to screen known ligands and tissue/cell extracts for activating ligands.

### EXAMPLE 7

### Microphysiometric Assays

Activation of a wide variety of secondary messenger systems results in extrusion of small amounts of acid from a cell. The acid formed is largely as a result of the increased metabolic activity required to fuel the intracellular signaling process. The pH changes in the media surrounding the cell are very small but are detectable by the CYTOSENSOR microphysiometer (Molecular Devices Ltd., Menlo Park, Calif., USA). The CYTOSENSOR is thus capable of detecting the activation of a receptor which is coupled to an energy utilizing intracellular signaling pathway such as a G-protein coupled receptor of the invention.

### EXAMPLE 8

### Extract/cell Supernatant Screening

A large number of mammalian receptors exist for which there remains, as yet, no cognate activating ligand (agonist). Thus, active ligands for these receptors may not be included within the ligands banks identified to date. Accordingly, the 7TM receptor of the invention is also functionally screened (using calcium, cAMP, microphysiometer, oocyte electrophysiology, etc., as functional screens) against tissue extracts to identify natural ligands. Extracts that produce positive functional responses can be sequencially subfractionated until an activating ligand is isolated and identified.

### EXAMPLE 9

### Calcium and cAMP Functional Assays

7TM receptors which are expressed in HEK 293 cells have been shown to be coupled functionally to activation of PLC and calcium mobilization and/or cAMP stimulation or inhibition. Basal calcium levels in the HEK 293 cells in receptor-transfected or vector control cells were observed to be in the normal, 100 nM to 200 nM, range. HEK 293 cells expressing recombinant receptors are loaded with fura 2 and in a single day>150 selected ligands or tissue/cell extracts are evaluated for agonist induced calcium mobilization. Similarly, HEK 293 cells expressing recombinant receptors are evaluated for stimulation or inhibition of cAMP production using standard cAMP quantitation assays. Agonists presenting a calcium transient or cAMP fluctuation are tested in vector control cells to determine if the response is unique to the transfected cells expressing receptor.

### EXAMPLE 10

### Catalytic Properties of DCPLD

To determine the activity encoded by recombinant DCPLD gene, baculovirus-infected Sf9 cells are assessed using a standard headgroup release assay that measures the amount of tritiated headgroup (e.g. [³H]-choline) liberated by hydrolysis of the labeled substrate [³H]-PC. The assay procedure measures release of the choline headgroup from the radiolabeled PC and is based on a protocol previously described in Brown, Cell 75 (1993) 1137-1144. Using standard separation techniques, cytosolic and membrane fractions are prepared from uninfected Sf9 cells or Sf9 cells infected for 48 hours with the DCPLD expressing baculovirus vector. Sf9 cells infected with native baculovirus vector or PLC-expressing baculovirus (control) vector PLD activity levels are determined. To determine the function of DCPLD as a transphophatidylase, the assay is performed in the presence of primary alcohols, catalyzing the transfer of the phosphatidyl group from an appropriate substrate to the alcohol e.g. ethanol, and thus generating [³²P]-phosphatidylethanol.

### EXAMPLE 11

### RNA Regulation of DCPLD

To analyze changes in the levels of DCPLD activity during differentiation of dendritic cells induced by LPS, TNFα or IL-10, levels of DCPLD mRNA are quantified by RT-PCR. Total RNA is isolated from the dendritic cells at various time points after activation by the guanidine thiocyanate method (Chomczynski, Anal. Biochem. 162 [1992] 156-162). RNA is reverse transcribed by using random hexamer mixed primers. The number of amplification cycles is determined to individual primer sets in order to maintain exponential rate of product amplification. Amplified DNA fragments are subjected to electrophoresis on 1.5% agarose gel and visualized by ethidium bromide staining. The intensity of bands is quantified by a densitometer.

1. An isolated gene which is
a DCEPR gene encoding a polypeptide of SEQ ID NO:2, or of SEQ ID NO:4, or
a DCTMF gene encoding a polypeptide of SEQ ID NO:6, or of SEQ ID NO:8, or
a DCPLD gene encoding a polypeptide of SEQ ID NO:10, or
a DCIGR gene encoding a polypeptide of SEQ ID NO:12, or of SEQ ID NO:14, or of SEQ ID NO:16, or
a DCLYR gene encoding a polypeptide of SEQ ID NO:18, or
a DCLEC gene encoding a polypeptide of SEQ ID NO:20, or of SEQ ID NO:22; or splice variants thereof, including
a DCEPR/SPLICE 1 gene encoding a polypeptide of SEQ ID NO:4, or
a DCTMF/SPLICE 1 gene encoding a polypeptide of SEQ ID NO:8, or
a DCIGR/SPLICE 1 gene encoding a polypeptide of SEQ ID NO:14, or
a DCIGR/SPLICE 2 gene encoding a polypeptide of SEQ ID NO:16, or
a DCLEC/SPLICE 1 gene encoding a polypeptide of SEQ ID NO:22;
or an isolated
DCEPR gene of SEQ ID NO:1, or
DCTMF gene of SEQ ID NO:5, or
DCPLD gene of SEQ ID NO:9, or
DCIGR gene of SEQ ID NO:11, or
DCLYR gene SEQ ID NO:17, or
DCLEC gene of SEQ ID NO:19, or splice variants thereof, including
a DCEPR/SPLICE 1 gene of SEQ ID NO:3, or
a DCTMF/SPLICE 1 gene of SEQ ID NO:7, or
a DCIGR/SPLICE 1 gene of SEQ ID NO:13, or
a DCIGR/SPLICE 2 gene of SEQ ID NO:15, or
a DCLEC/SPLICE 1 gene of SEQ ID NO:21.

2. An isolated polypeptide of SEQ ID NO:2, or of SEQ ID NO:4, or of SEQ ID NO:6, or of SEQ ID NO:8, or of SEQ ID NO:10, or of SEQ ID NO:12, or of SEQ ID NO:14, or of SEQ ID NO:16, or of SEQ ID NO:18, or of SEQ ID NO:20, or SEQ ID NO:22.

3. A vector comprising a gene according to claim 1.

4. An expression system comprising a DNA or RNA molecule isolated from the natural environment, wherein said expression system or part thereof is capable of producing a polypeptide according to claim 2 when said expression system or part thereof is present in a compatible host cell.

5. An isolated host cell comprising an expression system according to claim 4.

6. A recombinant host cell produced by transforming or transfecting a host cell with the expression system according to claim 4 such that the host cell, under appropriate culture conditions, produces a polypeptide according to claim 2.

7. An isolated phospholipid degrading enzyme derived from dendritic cells.

8. A diagnostic kit for a disease or susceptibility to a disease, comprising as a main component
a) a gene according to claim 1, or
b) a nucleotide sequence complementary to that of (a), or
c) a polypeptide according to claim 2, or
d) an antibody to a polypeptide according to claim 2.

9. An antibody against a polypeptide according to claim 2.

10. An immunological/vaccine formulation (composition) which, when introduced into a mammalian host, induces an immunological response in that mammal to a polypeptide according to claim 2, wherein the composition comprises a polypeptide according to claim 2 or a gene according to claim 1.

11. A screening assay for identifying an agonist or an antagonist of a polypeptide according to claim 2 which assay comprises as a main component
a) a polypeptide according to claim 2, or
b) a recombinant cell expressing a polypeptide according to claim 2, or
c) a cell membrane expressing a polypeptide according to claim 2, or
d) an antibody to a polypeptide according to claim 2.

12. A method of identifying an agonist or antagonist of a polypeptide according to claim 2, which comprises
A) contacting
a) a polypeptide according to claim 2, or
b) a recombinant cell expressing a polypeptide according to claim 2, or
c) a cell membrane expressing a polypeptide according to claim 2, or
d) an antibody to a polypeptide according to claim 2 with a candidate compound;
B) determining the effect of the candidate compound on any of a), b), c) or d); and
C) choosing an agonist or antagonist determined in step B).

13. An antagonist or an agonist of a polypeptide according to claim 2 which is characterized in that said antagonist or agonist can be provided by the following method steps:
A) contacting
a) a polypeptide according to claim 2, or
b) a recombinant cell expressing a polypeptide according to claim 2, or
c) a cell membrane expressing a polypeptide according to claim 2, or
d) an antibody to a polypeptide according to claim 2 with a candidate compound;
B) determining the effect of the candidate compound on any of a), b), c) or d); and
C) choosing an agonist or antagonist determined in step B).

14. An antagonist or an agonist according to claim 13 for use as a pharmaceutical.

15. A soluble form of a polypeptide according to claim 2 for use as a pharmaceutical.

16. A pharmaceutical composition comprising an agonist or an antagonist according to claim 13 or a soluble form of a polypeptide according to claim 2 in combination with pharmaceutically acceptable excipients/carriers.

17. A method of treating abnormal conditions related to both an excess of and insufficient level of expression of a gene according to claim 1; or related to both an excess of and insufficient activity of a polypeptide according to claim 2, comprising administering a therapeutically effective amount of an agonist or antagonist according to claim 13, or a therapeutically amount of a soluble form of a polypeptide according to claim 2 to a subject in need of said treating.
